(19) United States
(12) Patent Application Publication
(10) Pub. No.: US 2023/0090945 A1
(43) Pub. Date: Mar. 23, 2023

(54) Adeno-Associated Viral (AAV)-Mediated Sgpl1 Gene Therapy For Treatment Of Sphingosine-1-Phosphate Lyase Insufficiency Syndrome (SPLIS)

(71) Applicants: Children's Hospital & Research Center at Oakland, Oakland, CA (US); The Regents of the University of California, Oakland, CA (US)

(72) Inventors: Julie D. Saba, Oakland, CA (US); Piming Zhao, Jiangsu (CN)

(21) Appl. No.: 17/904,565

(22) PCT Filed: Feb. 18, 2021
(86) PCT No.: PCT/US2021/018613

§ 371 Date: Aug. 18, 2022

Related U.S. Application Data
(60) Provisional application No. 62/979,252, filed on Feb. 20, 2020.

(51) Int. Cl. A61K 48/00; C12N 9/88

(52) U.S. Cl. CPC A61K 48/005; C12N 9/88; C12Y 401/02027; C12N 2750/14143; A61K 38/00

(57) ABSTRACT

The present disclosure provides methods and compositions for transferring a gene encoding sphingosine-1-phosphate lyase (SPL) in a subject in need thereof by using recombinant AAV virions. The subject may have or may develop SPL insufficiency syndrome (SPLIS). The subject may be identified on the basis of a genetic test and/or SPLIS symptoms. The methods and compositions are also useful in lowering circulating sphingosine-1-phosphate levels in a subject having elevated S1P levels.

### CROSS-REFERENCE TO RELATED APPLICATIONS

This application claims the benefit of U.S. Provisional Application No. 62/979,252, filed Feb. 20, 2020, which application is incorporated herein by reference in its entirety.

### INTRODUCTION

SGPL1 gene encodes the vitamin B6-dependent enzyme sphingosine-1-phosphate lyase (SPL, also known as, S1PL), which catalyzes the irreversible degradation of the bioactive sphingolipid sphingosine-1-phosphate (S1P) in the final step of sphingolipid degradation (Expert Opin Ther Targets. 2009; 13(8):1013-25; J Lipid Res. 2019; 60(3):456-63). S1P serves as a ligand for a family of ubiquitously expressed G protein-coupled receptors (S1PRs). The S1PRs control actin cytoskeleton organization, cell migration, morphology and cell survival by signaling through downstream targets including MAPK, AKT and Rho GTPases (J Recept Signal Transduct Res. 2017; 37(5):437-46). S1P is generated from sphingosine by sphingosine kinases (SphK1/2) via a phosphorylation event that can be reversed by lipid- and S1P-specific phosphatases (Sgpp1/2) (Biochim Biophys Acta Mol Cell Biol Lipids. 2018; 1863(11):1413-22; J Lipid Res. 2015; 56(11):2048-60). However, only SPL can irreversibly degrade S1P. Thus, SPL is a critical regulator of S1P levels in blood and tissues (Expert Opin Ther Targets. 2009; 13(8):1013-25).

How a loss of SPL function might manifest in humans has remained unknown until very recently. NPHS14, a previously unrecognized inborn error of metabolism was identified in 2017 to be caused by recessive mutations (i.e., bi-allelic pathogenic variants) in SGPL1 (J Clin Invest. 2017; 127(3):912-28). Over the last two years, other groups have reported patients with SGPL1 mutations and similar presentations (J Clin Invest. 2017; 127(3):942-53; Hum Mutat. 2017; 38(4):365-72; Neurology. 2017; 88(6):533-42; Clinical Kidney Journal. 2017; 1-6; Brain Dev. 2018; 40(6):480-3; J Clin Endocrinol Metab. 2019 May 1; 104(5):1484-1490; Pediatr Nephrol. 2019; 34(1):77-9). Patients affected by NPHS14, which has been renamed SPL insufficiency syndrome (SPLIS) (Adv Biol Regul 2019 71:128-140) exhibit one or more major disease features including steroid-resistant nephrotic (protein spilling) syndrome with focal segmental glomerulosclerosis (FSGS) pathology, neurological defects, ichthyosis, lymphopenia and primary adrenal insufficiency (Adv Biol Regul 2019; 71:128-40). A wide range of severity is observed, with some patients dying in utero, others in infancy, while still others have presented late in the first decade of life and are living into adulthood, albeit requiring dialysis or kidney transplantation.

Thus, there is a need for methods for treating SPLIS. The present disclosure fulfills this and other needs.

### SUMMARY

The present disclosure provides methods and compositions for transferring a gene encoding sphingosine-1-phosphate lyase (SPL) into a subject in need thereof by using recombinant AAV virions. The subject may have or may develop SPL insufficiency syndrome (SPLIS). The subject may be identified on the basis of a genetic test, blood test for SPL concentration/activity, measurement of circulating S1P, and/or SPLIS symptoms, e.g., lymphopenia. In certain aspects, the subject has SPLIS and the transfer of the SPL encoding gene into the subject results in treatment of SPLIS in the subject. In certain aspects, the subject is susceptible to developing SPLIS and the transfer of the SPL encoding gene into the subject results in prevention of development of SPLIS in the subject. In certain aspects, the subject is susceptible to developing SPLIS and the transfer of the SPL encoding gene into the subject results in delay in onset of SPLIS and/or reduction in severity of SPLIS developed in the subject.

In certain aspects, the subject may have elevated levels of circulating S1P. The subject may have a condition other than SPLIS, e.g., inflammation, cancer, inflammatory bowel disease, and/or kidney disease and the transfer of the SPL encoding gene into the subject results in reduction of circulating S1P in the subject.

### BRIEF DESCRIPTION OF THE DRAWINGS

Included in the drawings are the following figures:

FIG. 1A. The SPL open reading frame encoded by SGPL1 is shown, with exons 1-15 indicated. The vitamin B6/PLP binding homology domain is shown by a box. Mutations found are shown in the schematic.

FIG. 1B. Major SPLIS features shown by percentage of patients.

FIG. 1C. Prenatal diagnoses associated with SPLIS (14 of 44 cases). AH, adrenal hemorrhage. Ca++, calcification.

FIG. 1D. Plasma S1P levels in five SPLIS patients.

FIG. 1E. MRI findings shown by age and pulse sequence in a single SPLIS patient.

FIG. 2A. Schematic of an AAV vector.

FIG. 2B. Skin fibroblasts derived from SPLIS patients (1-4) and one control were analyzed for SPL activity.

FIG. 2C. Skin fibroblasts derived from SPLIS patients (1-4) and one control were analyzed for cellular S1P content.

FIG. 2D. Infection of SPLIS fibroblasts with AAV2-hSPL or a 4-fold higher dose of AAV2-hSPLtRFP increased SPL expression well above endogenous levels.

FIG. 2E. Infection of SPLIS fibroblasts with AAV2-hSPL or a 4-fold higher dose of AAV2-hSPLtRFP increased SPL activity well above endogenous levels.

FIG. 3A. WB of kidney (Kid) and adrenal gland (AG) probed with anti-mSPL antibody. Right shows WB of liver probed with hSPL-specific antibody.

FIG. 3B. SPL activity in WT, SPL knock out “KO” and AAV-hSPL treated KO liver.

FIG. 3C. qRT-PCR of hSPL in AAV-hSPL treated KO mouse tissues.

FIG. 3D. Plasma sphingolipids in WT, SPL KO and AAV-hSPL-treated KO (AAV) mice.

FIG. 3E. Liver sphingolipids in WT, SPL KO and AAV-hSPL-treated KO (AAV) mice.

FIG. 3F. C14 and 16 ceramides in liver samples from WT, KO and treated mice.

FIG. 4A. Weight gain in KO, WT and AAV-hSPL treated KO pups.

FIG. 4B. Image of 22-day old WT and KO on left, WT and AAV-hSPL treated KO on right.

FIG. 4C. Survival of WT, KO and AAV-hSPL treated KO mice.

FIG. 4D. Serum albumin levels in WT, KO and early vs. late AAV-hSPL treated mice showing delayed disease progression.

FIG. 4E. Coomassie gel of urinary proteins.

FIG. 4F. Urine albumin levels corresponding to the gel.

FIG. 4G. Images of WT, KO and AAV-treated KO kidneys over time.

FIG. 4H. Blood cell indices show absolute lymphopenia (Abs Lymph), low % lymphocytes (% Lymph) and anemia with low red blood cell (RBC) counts, hemoglobin (HB) and hematocrit (HCT) in KO pups compared to WT.

FIGS. 5A-5C. Treatment of newborn Sgpl1 KO mice with AAV-SPL prolongs survival.

FIGS. 6A-6B. AAV-SPL treatment prevents the development of SPLIS nephrosis.

FIGS. 7A-7D. STAT3 activation and cytokine upregulation in SPLIS kidneys.

FIG. 8. Sgpl1 KO mice exhibit developmental delay, which is prevented by AAV-SPL.

FIGS. 9A-9B. Sgpl1 KO mice exhibit glucocorticoid deficiency.

FIG. 10. Plasma lipids of treated and untreated Sgpl1 KO mice.

FIG. 11. Comparison of SPL expression levels under the control of CAG vs. CMV promoter.

FIGS. 12A-12C. Schematics of vectors for introducing hSPL gene into a subject.

### DETAILED DESCRIPTION

The present disclosure provides methods and compositions for transferring a gene encoding sphingosine-1-phosphate lyase (SPL) in a subject in need thereof by using recombinant AAV virions. The subject may have or may develop SPL insufficiency syndrome (SPLIS). The subject may be identified on the basis of a genetic test and/or SPLIS symptoms.

Before exemplary embodiments of the present invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a rAAV virion” includes a plurality of such rAAV virions and reference to “the vector” includes reference to one or more vectors, “a mutation” refers to one or more mutations, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

“AAV” is an abbreviation for adeno-associated virus, and may be used to refer to the virus itself or derivatives thereof, such as, engineered AAVs. The term covers all subtypes and both naturally occurring and recombinant forms, except where required otherwise. The abbreviation “rAAV” refers to recombinant adeno-associated virus, also referred to as a recombinant AAV vector (or “rAAV vector”). The term “AAV” includes all AAV serotypes as well as AAV vectors based on the combination of different serotypes (also referred to as “hybrid AAV vectors” or “pseudotype AAV vectors”). In one aspect, the AAV vector is an AAV Vector with a serotype selected from AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, AAV-7m8 and combinations thereof. See, e.g., Mori et al. (2004) Virology 330:375. The synthetic AAV variant AAV-7m8 is described, for example, in Dalkara et al. See Transl Med 2013, 5 (189): 189ra76. The term “AAV” also includes chimeric AAV. “Primate AAV” refers to AAV isolated from a primate, “non-primate AAV” refers to AAV isolated from a non-primate mammal, “bovine AAV” refers to AAV isolated from a bovine mammal (e.g., a cow), etc. AAV also encompasses mixed serotype capsids, AAV-PHP.B, AAV-PHP.B2, AAV-PHP.B3, AAV-PHP.A, AAV-PHP.eB, AAV-PHP.eS, evolved capsids that are less immunogenic to mice and humans, and variants thereof.

An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two AAV inverted terminal repeat sequences (ITRs). The term rAAV vector encompasses both rAAV vector particles and rAAV vector plasmids.

An “AAV virus” or “AAV viral particle” or “rAAV vector particle” refers to a viral particle composed of at least one AAV capsid protein (typically by all of the capsid proteins of a wild-type AAV) and an encapsidated polynucleotide rAAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

“Packaging” refers to a series of intracellular events that result in the assembly and encapsidation of an AAV particle.

AAV “rep” and “cap” genes refer to polynucleotide sequences encoding replication and encapsidation proteins of adeno-associated virus. AAV rep and cap are referred to herein as AAV “packaging genes.”

A “helper virus” for AAV refers to a virus that allows AAV (e.g. wild-type AAV) to be replicated and packaged by a mammalian cell. A variety of such helper viruses for AAV are known in the art, including adenoviruses, herpesviruses and poxviruses such as vaccinia. The adenoviruses encompass a number of different subgroups, although Adenovirus type 5 of subgroup C is most commonly used. Numerous adenoviruses of human, non-human mammalian and avian origin are known and available from depositories such as the ATCC. Viruses of the herpes family include, for example, herpes simplex viruses (HSV) and Epstein-Barr viruses (EBV), as well as cytomegaloviruses (CMV) and pseudorabies viruses (PRV); which are also available from depositories such as ATCC.

“Helper virus function(s)” refers to function(s) encoded in a helper virus genome which allow AAV replication and packaging (in conjunction with other requirements for replication and packaging described herein). “Helper virus function” may be provided in a number of ways, including by providing helper virus or providing, for example, polynucleotide sequences encoding the requisite function(s) to a producer cell in trans.

An “infectious” virus or viral particle is one that comprises a polynucleotide component which it is capable of delivering into a cell for which the viral species is tropic. The term does not necessarily imply any replication capacity of the virus. As used herein, an “infectious” virus or viral particle is one that can access a target cell, can infect a target cell, and can express a heterologous nucleic acid in a target cell. Thus, “infectivity” refers to the ability of a viral particle to access a target cell, infect a target cell, and express a heterologous nucleic acid in a target cell. Infectivity can refer to in vitro infectivity or in vivo infectivity. Assays for counting infectious viral particles are described elsewhere in this disclosure and in the art. Viral infectivity can be expressed as the ratio of infectious viral particles to total viral particles. Total viral particles can be expressed as the number of viral genome (vg) copies. The ability of a viral particle to express a heterologous nucleic acid in a cell can be referred to as “transduction.” The ability of a viral particle to express a heterologous nucleic acid in a cell can be assayed using a number of techniques, including assessment of a marker gene, such as a green fluorescent protein (GFP) assay (e.g., where the virus comprises a nucleotide sequence encoding GFP), where GFP is produced in a cell infected with the viral particle and is detected and/or measured; or the measurement of a produced protein, for example by an enzyme-linked immunosorbent assay (ELISA). Viral infectivity can be expressed as the ratio of infectious viral particles to total viral particles. Methods of determining the ratio of infectious viral particle to total viral particle are known in the art.

A “replication-competent” virus (e.g. a replication-competent AAV) refers to a phenotypically wild-type virus that is infectious, and is also capable of being replicated in an infected cell (i.e. in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 10² rAAV particles, less than about 1 rcAAV per 10⁴ rAAV particles, less than about 1 rcAAV per 10⁸ rAAV particles, less than about 1 rcAAV per 10¹² rAAV particles, or no rcAAV).

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST or FASTA. Of particular interest are alignment programs that permit gaps in the sequence. Of interest is the BestFit program using the local homology algorithm of Smith Waterman (Advances in Applied Mathematics 2: 482-489 (1981) to determine sequence identity.

A “gene” refers to a polynucleotide containing at least one open reading frame that is capable of encoding a particular protein after being transcribed and translated.

“Recombinant,” as applied to a polynucleotide means that the polynucleotide is the product of various combinations of cloning, restriction or ligation steps, and other procedures that result in a construct that is distinct from a polynucleotide found in nature. A recombinant virus is a viral particle comprising a recombinant polynucleotide. The terms respectively include replicates of the original polynucleotide construct and progeny of the original virus construct.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

An “expression vector” is a vector comprising a region which encodes a polypeptide of interest, and is used for effecting the expression of the protein in an intended target cell. An expression vector also comprises control elements operatively linked to the encoding region to facilitate expression of the protein in the target. The combination of control elements and a gene or genes to which they are operably linked for expression is sometimes referred to as an “expression cassette,” a large number of which are known and available in the art or can be readily constructed from components that are available in the art.

“Heterologous” means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter. Thus, for example, an rAAV that includes a heterologous nucleic acid encoding a heterologous gene product is an rAAV that includes a nucleic acid not normally included in a naturally-occurring, wild-type AAV, and the encoded heterologous gene product is a gene product not normally encoded by a naturally-occurring, wild-type AAV.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: a) inhibiting the disease, i.e., arresting or slowing its development; and (b) relieving the disease, i.e., causing regression of the disease. One form of treatment includes prevention of occurrence of a disease or delay in onset of the disease and/or reduced severity of the disease after onset of the disease in a subject which may be predisposed to the disease or at risk of acquiring the disease but has not yet been diagnosed as having it.

The terms “individual,” “host,” “subject,” and “patient” are used interchangeably herein, and refer to a mammal, including, but not limited to, human and non-human primates, including simians and humans; mammalian sport animals (e.g., horses, camels, etc.); mammalian farm animals (e.g., sheep, goats, cows, etc.); mammalian pets (dogs, cats, etc.); and rodents (e.g., mice, rats, etc.). In some cases, the individual is a human.

The term “pharmaceutically acceptable” refers to a non-toxic material which preferably does not interfere with the action of the active ingredient of the pharmaceutical composition. In particular, the term “pharmaceutically acceptable” means that the subject substance has been approved by a governmental regulatory agency for use in animals, and particularly humans, or in U.S. Pat. Pharmacopoeia, European Pharmacopoeia or other recognized pharmacopoeias for use in animals and in particular humans.

### Methods

The present disclosure provides a method for treating sphingosine-1-phosphate lyase (SPL) insufficiency syndrome (SPLIS) in a subject. The method involves transfer of a nucleic acid encoding SPL via recombinant AAV virions into a subject in need thereof. The present disclosure also encompasses prevention of occurrence of SPLIS in a subject at risk for developing SPLIS. Various steps and aspects of the method will now be described in greater detail below.

SGPL1 gene encodes the vitamin B6-dependent enzyme SPL, which catalyzes the irreversible degradation of the bioactive sphingolipid sphingosine-1-phosphate (S1P) in the final step of sphingolipid degradation. As discussed in detail in the Examples section of the specification, SPLIS patients harbor inactivating mutations in SGPL1 gene and exhibit one or more major disease features including steroid-resistant nephrotic (protein spilling) syndrome with focal segmental glomerulosclerosis (FSGS) pathology, neurological defects (developmental delay or regression, ataxia, cranial nerve defects, seizures, and peripheral neuropathy), ichthyosis, cranial nerve palsies, bony abnormalities, hypocalcemia, hypothyroidism, gonadal defects, lymphopenia/immunodeficiency and primary adrenal insufficiency with a wide range of severity. SPLIS patients have reduced life span with some patients dying in utero, others in infancy. SPLIS patients can present late in the first decade of life, and may survive to adulthood, albeit requiring dialysis or kidney transplantation.

A subject who may be treated with the methods disclosed herein may be a newborn, an infant, a toddler, a child, a teenager, or an adult. In certain aspects, the subject may be a fetus in utero. The subject may exhibit one or more symptoms of SPLIS and/or have a mutation associated with lack of sufficient SPL activity. In some aspects, a subject receiving a rAAV virion according to the present disclosure may be treated prior to onset of symptoms of SPLIS, based on, e.g., a genetic test and/or a blood test. An inactivating SPL mutation that results in significant lack of enzyme activity may be one or more of nonsense mutations, splicing defects, and missense mutations. In certain aspects, a subject in need of treatment for SPLIS may be asymptomatic but may be identified by low to undetectable SPL abundance and/or activity, e.g., in skin fibroblasts obtained from the subject by, e.g., a skin biopsy. In certain aspects, cells from a buccal swab may be assayed to determine low SPL level or activity. In certain aspects, a subject may be diagnosed as having or at risk of developing SPLIS prior to the onset of symptoms by next generation DNA sequence analysis of whole genome or whole exome in infants with lymphopenia or in a sibling of a previously diagnosed SPLIS patient. In certain aspects, lymphopenia may be persistent idiopathic lymphopenia diagnosed at birth,

In certain aspects, the method for treating or preventing SPL insufficiency syndrome (SPLIS) in a subject involves administering to the subject a recombinant adeno-associated viral (rAAV) virion comprising a nucleic acid encoding sphingosine-1-phosphate lyase (SPL), wherein administering the rAAV virion results in expression of SPL in the subject thereby treating the SPLIS in the subject. As discussed above, the subject may or may not exhibit symptoms of SPLIS. For example, the subject may be identified as in need for treatment based on presence of an inactivating SPL mutation and/or low to undetectable SPL abundance and/or activity. Thus, the disclosed method can involve prophylactic treatment of a subject to prevent SPLIS symptoms from developing. As such the disclosed method encompasses treatment of a subject having an inactivating SPL mutation and/or low to undetectable SPL abundance and/or activity. SPL level and/or activity may be measured in cells obtained from the subject, such as fibroblasts from a skin biopsy or a buccal swab. Level and/or activity lower than a normal level may indicate that the subject has a SPL inactivating mutation. In newborns, detection of lymphopenia may indicate that the newborn may have a SPL inactivating mutation. In certain aspects, the presence of the mutation may be confirmed by sequencing the SPL gene in the subject. SPL inactivating mutations are known. An SPL inactivating mutation may be homozygous or heterozygous. In certain aspects, the SPL inactivating mutation may be a frameshift mutation, truncation, a mutation resulting in splicing defect, a substitution, deletion, mutation in 5′ untranslated region of the gene that prevent expression, mutations in introns, exons, or 3′-untranslated region of the mRNA that promote mRNA degradation. In certain aspects, the mutation may be a frameshift mutation (e.g., Gly360Alafs*49, Ser3Lysfs*11, Ser65Argfs*6, Arg278Glyfs*17, Leu312Phefs*30, or Phe411Leufs*56,), a truncation (e.g., Tyr331*, Ser361*, or Arg505*), a mutation resulting in splicing defect, a substitution, e.g., a substitution at residue 353, replacing the cofactor binding lysine with an arginine (Lys353Arg), a Arg222Gln substitution, a Gly360Val substitution, a Phe290Leu substitution, a Ser202Leu substitution, a Tyr416Cys substitution, a Cys285Tyr substitution, a Tyr15Cys substitution, a Lys353Arg substitution, a Ile184Thr substitution, a Arg340Trp substitution, a Ser346Ile substitution, or a combination thereof. In certain aspects, a subject may have more than one mutation in the SPL gene.

The rAAV virion may be administered in a therapeutically effective amount. A “therapeutically effective amount” will fall in a relatively broad range that can be determined through experimentation and/or clinical trials. For example, a therapeutically effective dose can be on the order of from about 10⁶ to about 10¹⁵ of the rAAV virions, e.g., from about 10⁸ to 10¹² rAAV virions, from about 10⁶ viral genomes (vg) to about 10¹⁵ vg of the rAAV virions, e.g., from about 10⁸ vg to 10¹² vg. Other effective dosages can be readily established by one of ordinary skill in the art through routine trials establishing dose response curves.

In some embodiments, more than one administration (e.g., two, three, four or more administrations) may be employed to achieve the desired level of gene expression. In some cases, the more than one administration is administered at various intervals, e.g., daily, weekly, twice monthly, monthly, every 3 months, every 6 months, yearly, etc. In some cases, multiple administrations are administered over a period of time from 1 month to 2 months, from 2 months to 4 months, from 4 months to 8 months, from 8 months to 12 months, from 1 year to 2 years, from 2 years to 5 years, or more than 5 years.

In some embodiments the AAV serotype is selected from: AAV1, AAV2, AAV3, AAV4, AAV5, AAV6, AAV7, AAV8, and AAVrh.10. In other embodiments the AAV serotype is a variant of an AAV serotype is selected from: AAV1, AAV2, AAV3, AAV4, AAV5, AAV6, AAV7, AAV8, and AAVrh.10. In some aspects, the rAAV virion comprises AAV serotype 9 capsid proteins. The nucleic acid encoding SPL is flanked by AAV inverted terminal repeats (ITRs), e.g., AAV2 ITRs or AAV9 ITRs.

In certain aspects, the subject may be human and the SPL may be a human SPL. The nucleic acid present in the AAV may encode a human SPL having an amino acid sequence that is at least 80%, 85%, 90%, 95%, 96%, 97%, 98%, or 99% or a 100% identical to the amino acid sequence:

(SEQ ID NO: 1)
MPSTDLLMLKAFEPYLEILEVYSTKAKNYVNGHCTK
YEPWQLIAWSVVWTLLIVWGYEFVFQPESLWSRFKK
KCFKLTRKMPIIGRKIQDKLNKTKDDISKNMSFLKV
DKEYVKALPSQGLSSSAVLEKLKEYSSMDAFWQEGR
ASGTVYSGEEKLTELLVKAYGDFAWSNPLHPDIFPG
LRKIEAEIVRIACSLFNGGPDSCGCVTSGGTESILM
ACKAYRDLAFEKGIKTPEIVAPQSAHAAFNKAASYF
GMKIVRVPLTKMMEVDVRAMRRAISRNTAMLVCSTP
QFPHGVIDPVPEVAKLAVKYKIPLHVDACLGGFLIV
FMEKAGYPLEHPFDFRVKGVTSISADTHKYGYAPKG
SSLVLYSDKKYRNYQFFVDTDWQGGIYASPTIAGSR
PGGISAACWAALMHFGENGYVEATKQIIKTARFLKS
ELENIKGIFVFGNPQLSVIALGSRDFDIYRLSNLMT
AKGWNLNQLQFPPSIHFCITLLHARKRVAIQFLKDI
RESVTQIMKNPKAKTTGMGAIYGMAQTTVDRNMVAE
LSSVFLDSLYSTDTVTQGSQMNGSPKPH.

As used herein, human SPL refers to a polypeptide having the amino acid sequence set forth above as SEQ ID NO:1, a functional fragment thereof, or a polypeptide having SPL activity and having an amino acid sequence at least 80% identical (e.g., at least 85%, at least 90%, at least 95%, at least 96%, at least 97%, at least 98%, or at least 99%) to the amino acid sequence set forth in SEQ ID NO:1. In certain aspects, the nucleic acid encoding the human SPL may be a codon-optimized sequence, where the codons are codons from highly expressed human genes.

In certain aspects, the SPL may be a soluble human SPL that lacks the transmembrane region of the full-length SPL. For example, the soluble human SPL may lack an N-terminal sequence, such as, the first 66 N-terminal amino acids present in SEQ ID NO:1.

Treatment of SPLIS in a subject in need thereof may result in a decrease of sphingolipids, S1P, and/or sphingosine in blood of the patient. Treatment of SPLIS in a subject in need thereof may result in a decrease of cholesterol and triglyceride levels in the subject. Treatment of SPLIS in a subject in need thereof may result in a reduction in C14 and 16 ceramides in liver of the subject. The decrease may be at least 10% or more (e.g., 20%, 30%, 40%, 50%, or more) as compared to prior to the treatment. The treatment may increase life span of the subject by at least 1 year or more, e.g., at least 5 years, 10 years, 15 years, or more as compared to subjects with SPLIS not receiving the treatment.

The presently disclosed rAAV finds use in reducing circulating S1P level in a subject in need thereof. Circulating S1P level may be measured in a fluid sample from the subject, such as, a blood sample or a fraction thereof, e.g., serum or plasma. The measured level may be compared to a normal level to determine whether a subject has increased circulating S1P level. The measured level may be compared to a threshold level that is higher than the normal level to determine whether a subject has increased circulating S1P level. The normal and threshold S1P levels may be determined based on S1P measurement in age matched and/or gender matched healthy subjects. The subject may have SPLIS or may be susceptible to developing SPLIS or may have another condition such as inflammation, cancer, inflammatory bowel disease, or kidney disease. The administration of the rAAV may result in an expression level of SPL that is at least 10% of the normal SPL level and may result in a 10% or higher reduction in the circulating S1P level as compared to prior to the treatment.

In certain aspects, in addition to reducing circulating levels of S1P, the administration of the rAAV of the present disclosure may reduce S1P level in tissues, such as, liver, kidney, brain and the like. In certain aspects, the rAAV of the present disclosure may reduce S1P level in tissues, such as, liver, by at least 10% (e.g., at least 20%, at least 30%, at least 40%, at least 50%, at least 60%, or more) as compared to S1P level prior to the treatment with the rAAV.

The administration of the rAAV of the present disclosure may preserve and/or improve kidney function. Kidney function can be assessed by measuring serum albumin and/or urine albumin to creatinine ratio (ACR). Treatment with the rAAV may reduce urine ACR by at least 10% (e.g., 20%, 30%, 40% or more) as compared to urine ACR prior to the treatment with the rAAV. Treatment with the rAAV may increase serum albumin level by at least 10% (e.g., 20%, 30%, 40% or more) as compared to serum albumin level prior to the treatment with the rAAV.

The administration of the rAAV of the present disclosure may reduce or prevent STAT3 pathway activation and elevated pro-inflammatory and fibrogenic cytokines. For example, the reduction in STAT3 pathway activation may be at least 10% (e.g., 20%, 30%, 40% or higher) as compared to the activation prior to the treatment with the rAAV. The reduction in elevated pro-inflammatory and fibrogenic cytokines may be at least 10% (e.g., 20%, 30%, 40% or higher) as compared to the level of pro-inflammatory and fibrogenic cytokines prior to the treatment with the rAAV.

Recombinant AAVs: Production Methods

Methods for obtaining recombinant AAVs having a desired capsid protein are well known in the art. (See, for example, US 2003/0138772, the contents of which are incorporated herein by reference in their entirety). Typically, the methods involve culturing a host cell which contains a nucleic acid sequence encoding an AAV capsid protein or fragment thereof, a functional rep gene, a recombinant AAV vector composed of. AAV inverted terminal repeats (ITRs) and a therapeutic transgene: and sufficient helper functions to permit packaging of the recombinant AAV vector into the AAV capsid proteins.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the invention may be delivered to the packaging host cell using any appropriate genetic element (vector). The selected genetic element may be delivered by any suitable method, including those described herein. The methods used to construct any embodiment of this invention are known to those with skill in nucleic acid manipulation and include genetic engineering, recombinant engineering, and synthetic techniques.

Typically, the recombinant AAVs are produced by transfecting a host cell with an recombinant AAV vector (comprising a therapeutic transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable wild-type AAV virions (i.e., AAV virions containing functional rep and cap genes). The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpes virus (other than herpes simplex virus type-1), and vaccinia virus.

In some aspects, the invention provides transfected host cells. The term “transfection” is used to refer to the uptake of foreign DNA by a cell, and a cell has been “transfected” when exogenous DNA has been introduced inside the cell membrane A number of transfection techniques are generally known in the art. Transfection may be achieved for example by infecting a cell with a rAAV harboring a rAAV vector A “host cell” refers to any cell that harbors, or is capable of harboring, a substance of interest. Often a host cell is a mammalian cell. A host cell may be used as a recipient of an AAV helper construct, an AAV transgene plasmid, e.g., comprising a promoter operably linked with a S1PL encoding gene, an accessory function vector, or other transfer DNA associated with the production of recombinant AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter.

The foregoing methods for packaging recombinant vectors in desired AAV capsids to produce the rAAVs of the present disclosure are not meant to be limiting and other suitable methods will be apparent to the skilled artisan.

Recombinant AAV Vectors

“Recombinant AAV (rAAV) vectors” of the present disclosure are typically composed of, at a minimum, a therapeutic transgene, e.g., encoding a SPL, and its regulatory sequences and 5 and 3′ AAV inverted terminal repeats (ITRs). It is this recombinant AAV vector which is packaged into a capsid protein and delivered to a subject. The nucleic acid sequence encoding a S1PL is operatively linked to regulatory components in a manner which permits transgene transcription, translation, and/or expression in a cell.

The nucleic acid encoding a SPL, may have the following sequence or may have a sequence at least 50% identical (e.g., at least 60%, at least 70%, at least 80%, at least 85%, at least 90%, or at least 95% identical) to the following nucleic acid sequence.

(SEQ ID NO: 2)
ATGCCTAGCACAGACCTTCTGATGTTGAAGGCCTTT
GAGCCCTACTTA
GAGATTTTGGAAGTATACTCCACAAAAGCCAAGAAT
TATGTAAATGGACATTGCACCAAGTATGAGCCCTGG
CAGCTAATTGCATGGAGTGTCGTGTGGACCCTGCTG
ATAGTCTGGGGATATGAGTTTGTCTTCCAGCCAGAG
AGTTTATGGTCAAGGTTTAAAAAGAAATGTTTTAAG
CTCACCAGGAAGATGCCCATTATTGGTCGTAAGATT
CAAGACAAGTTGAACAAGACCAAGGATGATATTAGC
AAGAACATGTCATTCCTGAAAGTGGACAAAGAGTAT
GTGAAAGCTTTACCCTCCCAGGGTCTGAGCTCATCT
GCTGTTTTGGAGAAACTTAAGGAGTACAGCTCTATG
GACGCCTTCTGGCAAGAGGGGAGAGCCTCTGGAACA
GTGTACAGTGGGGAGGAGAAGCTCACTGAGCTCCTT
GTGAAGGCTTATGGAGATTTTGCATGGAGTAACCCC
CTGCATCCAGATATCTTCCCAGGACTACGCAAGATA
GAGGCAGAAATTGTGAGGATAGCTTGTTCCCTGTTC
AATGGGGGACCAGATTCGTGTGGATGTGTGACTTCT
GGGGGAACAGAAAGCATACTGATGGCCTGCAAAGCA
TATCGGGATCTGGCCTTTGAGAAGGGGATCAAAACT
CCAGAAATTGTGGCTCCCCAAAGTGCCCATGCTGCA
TTTAACAAAGCAGCCAGTTACTTTGGGATGAAGATT
GTGCGGGTCCCATTGACGAAGATGATGGAGGTGGAT
GTGCGGGCAATGAGAAGAGCTATCTCCAGGAACACT
GCCATGCTCGTCTGTTCTACCCCACAGTTTCCTCAT
GGTGTAATAGATCCTGTCCCTGAAGTGGCCAAGCTG
GCTGTCAAATACAAAATACCCCTTCATGTCGACGCT
TGTCTGGGAGGCTTCCTCATCGTCTTTATGGAGAAA
GCAGGATACCCACTGGAGCACCCATTTGATTTCCGG
GTGAAAGGTGTAACCAGCATTTCAGCTGACACCCAT
AAGTATGGCTATGCCCCAAAAGGCTCATCATTGGTG
TTGTATAGTGACAAGAAGTACAGGAACTATCAGTTC
TTCGTCGATACAGATTGGCAGGGTGGCATCTATGCT
TCCCCAACCATCGCAGGCTCACGGCCTGGTGGCATT
AGCGCAGCCTGTTGGGCTGCCTTGATGCACTTCGGT
GAGAACGGCTATGTTGAAGCTACCAAACAGATCATC
AAAACTGCTCGCTTCCTCAAGTCAGAACTGGAAAAT
ATCAAAGGCATCTTTGTTTTTGGGAATCCCCAATTG
TCAGTCATTGCTCTGGGATCCCGTGATTTTGACATC
TACCGACTATCAAACCTGATGACTGCTAAGGGGTGG
AACTTGAACCAGTTGCAGTTCCCACCCAGTATTCAT
TTCTGCATCACATTACTACACGCCCGGAAACGAGTA
GCTATACAATTCCTAAAGGACATTCGAGAATCTGTC
ACTCAAATCATGAAGAATCCTAAAGCGAAGACCACA
GGAATGGGTGCCATCTATGGCATGGCCCAGACAACT
GTTGACAGGAATATGGTTGCAGAATTGTCCTCAGTC
TTCTTGGACAGCTTGTACAGCACCGACACTGTCACC
CAGGGCAGCCAGATGAATGGTTCTCCAAAACCACAC

In some cases, the nucleic acid encoding a human SPL may be a codon optimized version of the nucleotide sequence set forth in SEQ ID NO:2. For example, one or more codons in SEQ ID NO:2 may be replaced with codon(s) from highly expressed human genes to increase expression of the SPL in a human subject receiving the rAAV.

The AAV sequences of the vector typically comprise the cis-acting 5′ and 3′ inverted terminal repeat sequences. The ITR sequences are about 145 bp in length. An example of such a molecule is a “cis-acting” plasmid containing the nucleic acid sequence encoding a S1PL, in which the nucleic acid sequence encoding a S1PL and associated regulatory elements are flanked by the 5′ and 3′ AAV ITR sequences. The AAV ITR sequences may be obtained from any known AAV, including presently identified mammalian AAV types. The 5′ and 3′ AAV ITR sequences may be AAV9 5′ and 3′ ITR sequences, respectively, or AAV2 5′ and 3′ ITR sequences, respectively.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals: sequences that stabilize cytoplasmic mRNA, sequences that enhance translation efficiency (i.e., Kozak consensus sequence); sequences that enhance protein stability; and when desired, sequences that enhance secretion of the encoded product. A great number of expression control sequences including promoters which are native, constitutive, inducible and/or tissue-specific, are known in the art and may be utilized.

In some embodiments, the regulatory sequences impart tissue-specific gene expression capabilities. In some cases, the tissue-specific regulatory sequences bind tissue-specific transcription factors that induce transcription in a tissue specific manner such tissue-specific regulatory sequences (e.g., promoters, enhancers. etc.) are well known in the art.

In certain aspects, the promoter may be a CMV, a chicken beta actin promoter, human β-actin/CMV hybrid promoter, chicken β-actin/CMV hybrid promoter, CMV-actin-globin (CAG) hybrid promoter, Math Promoter, VGLUT3 promoter, parvalbumin promoter, calretinin promoter, calbindin 28 k promoter, prestin promoter, a liver specific promoter, e.g., albumin promoter, endogenous SGPL1 promoter for human SPL, and the like. In certain aspects, the human SGPL1 promoter may have a nucleotide sequence that extends from −200 to +100 of the 5′ region of the SGPL1 gene and has the following sequence:

(SEQ ID NO: 3)
acacacacacacagggcttcatcggctcagagagttgca
aacccagacccatgttctaggctgggctctgccaggaac
tgctaggagtcgcatctctctctggacctcagtttctt
cacgtgtatttgtgtattctctggggtggagctggcga
atagctggagacccctctagccctaccattttttgatt
ctaattaaccaaaaaggatattcgaggtccccgctaca
aattctgaacccttggcttcccctccaaactcccacac
aaactccaccccatcctgcctgtgtgtctttgggagga
tcatttccttctttggtcttggtgtccttgtttacagg
ttgagggatgataagagtcacctgatctgggcaagtca
ggccataaataaggcttgtatgtaaaggtgcctagcat
agttcttggcaaagcaaggatcagtcgatatgattgca
ttggtttgagcatctgggaagaatgagcagactgcata
ctgtgtgttagggtgtagggaggaatcagacgtgaccc
tgccctctagtcatctgggtgcacagattctagactaa
aggatgtgggggtggtaactaacaaggagatggagagg
aggtgtgcagttcctgggggatctgatgaagaattaaa
gcagaagggtaggagatggagaatgggaaggcatacct
gaaaccctaaagcaactcttgatgagtaggcattggca
ggaggccccagaacattttctgaccctcaaacactgag
aatgcatgtctgctggggaatagtagtgagggggctaa
gggtatgggggtgttcatgcctgagtcaggggctctgg
atggtagaaatgtctgggagttctgacggaatgggggt
gaggaggcctaaagataacctgttcatagtctttcagg
gccttaaccattatggctggggaagtgggaagtgccga
tggggtatgcagaggagggcagctgtgatagtacattt
catcctgaaagcactacatgtggataatgacggtagtg
atgatgccctaattttagtgtcatggcaacattttcca
aggggatggaattgggtaccctcattagtgtcattttt
gtttgagagatttcaagagtctatctaggtgtcatttt
aaaatctattgaaaaaaaatcttgaatgcaggtcactg
accagttggttccatgtaactgcactgccttctgctac
cactaaagcaatgttatgacaacctggacaagtcatgg
gaaacctatgtgagacnagttttttctttttctttttg
gagttgggggtctcactatgttgctcaggctgatctag
agctcctgggctcagacaaccctcccgcctcagcatcc
ccaagtgccgggattaccgatgtaagccactacaccca
gctgagacttagttttttcatctggaaatgaaggcgac
ctttgtgatcccagtgggcccttgcacttgtgtataag
gtgattcccagcactgctcatgaaagtagtgatggcag
agtagcctctgctctccattttatttttggggtggggg
ggggcagtggtggaggggaggatgaggatgatatttcc
attaaataagtttccttgttgcttttcttcaggcacca
ctgacctcgatggaaaaatgaagtccctggcccaggaa
agagacccaactctgtggctgttttggattagtttgta
caatgccctgcagacctactctctcaggaggctccaag
tgaccaaatgtgacaagaaagttttttgtecaaggcag
tttggaagagaagttggtgaccgtcgggataccacagc
cgccccaggacggcctggttgagacattcactggaggg
tctgggtgcagcccgctgcctggccggtaggcggcgcg
cacaggccgtggggcccgggtctgggcgtgcgcgcggc
tggtagcagcggggccgcgcacgccagggtccgggagc
cgggccggtgcccccggagccATTTCCGGGAGGGGCGA
GGCCGGCGGCTGCCGGGCCTCCAATCTCGGCGGCGGCG
GCGGCAACAGGGGAGCCTGGGTCTCGCGGCCTGCGAGT
CCGTCGCG

Sequence from −2000 to +100 where +1 is the start of transcription and sequence upstream of +1 is in lower case. Longer and shorter versions of the human SGPL1 promoter may also be used.

In certain aspects, the AAV vector is selected from the group consisting of AAV-PHP.B, AAV-PHP.eB, AAV-PHP.S and AAV-Anc80. In certain aspects, the AAV vector AAV-PHP.eB or AAV-PHP.S.

In certain aspects, the AAV vector is selected from the group consisting of AAV-8, AAV-9 and AAV-1/2

In certain aspects, the subject may be administered more than one type of AAV vector to target different tissues. For example, AAV-PHP.eB and AAV-PHP.S, capsids that efficiently transduce the central and peripheral nervous systems, respectively, may be used to target the SPL gene to the central and peipheral nervous systems. An exo-AAV vector may be used to target liver, retina, inner ear, and/or nervous system.

Recombinant AAV Virion Administration Methods

The rAAV virion may be delivered to a subject in compositions according to any appropriate methods known in the art. The rAAV virion, preferably suspended in a physiologically compatible carrier or a pharmaceutically acceptable excipient (i.e., in a composition), may be administered to a subject, such as, for example, a human, mouse, rat, cat, dog, sheep, rabbit, horse, cow, goat, pig, guinea pig, hamster, chicken, turkey, or a non-human primate (e.g., Macaque).

The rAAV virions are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal or intrahepatic delivery to the liver), oral, inhalation (including intranasal and intratracheal delivery), intraocular, intravenous, intramuscular, subcutaneous, intradermal, intrathecal, and other parental routes of administration. In certain circumstances it will be desirable to deliver the rAAV-based therapeutic constructs in suitably formulated pharmaceutical compositions disclosed herein either subcutaneously, intra-pancreatically, intranasally, parenterally, intravenously, intramuscularly, intrathecally, orally, intraperitoneally, or by inhalation. In certain aspects the administration may be into the brain, e.g., intraparenchymally, intracerebroventricularly, into cisterna magna or subpial).

Delivery of the rAAV virions to a mammalian subject may be by intravenous injection. In some embodiments, the mode of administration of rAAV virions is by portal vein injection. Administration into the bloodstream may be by injection into a vein, an artery, or any other vascular conduit. In some embodiments, administration of rAAV virions into the bloodstream is by way of isolated limb perfusion, a technique well known in the surgical arts, the method essentially enabling the artisan to isolate a limb from the systemic circulation prior to administration of the rAAV virions. Routes of administration may be combined, if desired.

Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

The compositions of the invention may comprise a rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes. In certain aspects, the composition may include an additional therapeutic agent for treatment for SPLIS.

In a further aspect, the present invention provides a viral vector or a virion of the present invention or a pharmaceutical composition of the present invention for use as a medicament.

In a further aspect, the present invention provides a viral vector or a virion of the present invention or a pharmaceutical composition of the present invention for use in a method of treating SPLIS, preventing SPLIS, or for reducing circulating SIP in a subject in need thereof.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. Optionally, the compositions of the present disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene expression required to achieve a therapeutic effect, the specific disease or disorder being treated, and the stability of the gene or RNA product. One of skill in the art can readily determine a rAAV virion dose range to treat a subject having a particular disease or disorder based on the aforementioned factors, as well as other factors that are well known in the art.

An effective amount of a rAAV is an amount sufficient to target infect an animal, target a desired tissue. In some embodiments, an effective amount of a rAAV is an amount sufficient to produce a stable somatic transgenic animal model. The effective amount will depend primarily on factors such as the species, age, weight, health of the subject, and the tissue to be targeted, and may thus vary among animal and tissue. For example, an effective amount of the rAAV is generally in the range of from about 1 ml to about 100 ml of solution containing from about 10⁹ to 10¹⁶ genome copies. In some cases, a dosage between about 10¹¹ to 10¹² rAAV genome copies is appropriate. In certain preferred embodiments, 10¹² rAAV genome copies is effective to target brain, heart, liver, and pancreatic tissues. In certain embodiments, the dosage of rAAV is 10¹⁰, 10¹¹, 10¹², 10¹³, or 10¹⁴ genome copies per kg. In certain embodiments, the dosage of rAAV is 10¹⁰, 10¹¹, 10¹², 10¹³, 10¹⁴, or 10¹⁵ genome copies per subject. In some cases, stable transgenic animals are produced by multiple doses of a rAAV.

In certain aspects, a therapeutically effective dose provides a level and/or activity of SPL in the subject that is at least 10% of the normal SPL level and/or activity, respectively (e.g., at least 20%, at least 30%, at least 40%, at least 45%, at least 50%, or more of the normal SPL level). In certain aspects, the level and/or activity of SPL may be measured in cells obtained from the subject, such as fibroblasts from a skin biopsy or a buccal swab. Normal SPL level and/or activity can be measured in age matched and/or gender matched healthy subjects, e.g., skin fibroblasts from healthy subjects.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc.

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration in this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion. Some variation in dosage will necessarily occur depending on the condition of the host. The person responsible for administration will, in any event, determine the appropriate dose for the individual host.

Sterile injectable solutions are prepared by incorporating the active rAAV virions in the required amount in the appropriate solvent with various of the other ingredients enumerated herein, as required, followed by filtered sterilization. The rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, vesicles, and the like, may be used for the introduction of the compositions of the present invention into suitable host cells. In particular, the rAAV vector delivered transgenes may be formulated for delivery either encapsulated in a lipid particle, a liposome, a vesicle, a nanosphere, or a nanoparticle or the like.

Combination Therapy

In certain aspects, the subject having SPLIS may receive an additional therapy for treating SPLIS. The additional therapy may be administered prior to, simultaneously with, or after administration of the rAAV of the present disclosure. The additional therapy may include administration of an agent, e.g., a compound for treating SPLIS. Such an agent may be Vitamin B6 Cofactor, adrenal steroids, anti-S1P antibodies, SphK1/2 inhibitors, and the like

In certain aspects, the subject having increased circulating levels of S1P having inflammation, cancer, inflammatory bowel disease, or kidney disease may receive an additional therapy decreasing S1P, such as, Vitamin B6 Cofactor Supplementation, adrenal steroids, anti-S1P antibodies, SphK1/2 inhibitors, and the like.

### EXEMPLARY NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A method for treating SPL insufficiency syndrome (SPLIS) in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the SPLIS in the subject.

2. The method of aspect 1, wherein the subject has an increased level of plasma sphingosine-1-phosphate (S1P) and the administering results in at least a 5% reduction in plasma sphingosine-1-phosphate (S1P) in the subject.

3. The method of aspect 1 or 2, wherein the subject has an increased level of C14 and 16 ceramides in the liver and the administering results in at least a 5% reduction in C14 and 16 ceramides in liver of the subject.

4. The method of any one of aspects 1-3, wherein the subject has hypoalbuminemia and the administering results in at least a 5% reduction in hypoalbuminemia in the subject.

5. The method of any one of aspects 1-4, wherein the subject has albuminuria and the administering results in at least a 5% reduction in albuminuria in the subject.

6. The method of any one of aspects 1-5, wherein the expression of SPL in the subject after the administering is at least 10% of the normal SPL level.

7. The method of any one of aspects 1-6, wherein the subject has lymphopenia and is diagnosed as having SPLIS based on sequencing of the SGPL1 gene.

8. The method of aspect 7, wherein the subject is a newborn.

9. A method for preventing a subject from developing SPL insufficiency syndrome (SPLIS), the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing the subject from developing SPLIS.

10. The method of aspect 9, wherein the subject has an inactivating mutation in SGPL1 gene.

11. The method of aspect 9 or 10, wherein the subject is a fetus in utero, a newborn, an infant, a toddler, or a child.

12. The method of any one of aspects 9-11, wherein the expression of SPL in the subject after the administering is at least 10% of the normal SPL level.

13. A method for reducing circulating sphingosine-1-phosphate (S1P) level in a subject having increased circulating S1P level, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of SPL in the subject thereby reducing the circulating S1P level in the subject.

14. The method of aspect 13, wherein the subject has or is at risk for developing SPL insufficiency syndrome (SPLIS).

15. The method of aspect 13, wherein the subject has inflammation.

16. The method of aspect 13, wherein the subject has cancer.

17. The method of aspect 13, wherein the subject has inflammatory bowel disease.

18. The method of aspect 13, wherein the subject has kidney disease.

19. The method of any one of aspects 1-18, wherein the rAAV virion comprises AAV serotype 9 capsid proteins.

20. The method of any one of aspects 1-18, wherein the rAAV virion comprises AAV-PHP.eb virions.

21. The method of any one of aspects 1-18, wherein the rAAV virion comprises AAV-PHP.S virions.

22. The method of any one of aspects 1-21, wherein the nucleic acid encoding SPL is flanked by AAV inverted terminal repeats (ITRs).

23. The method of any one of aspects 1-22, wherein the subject is a human and the SPL is a human SPL.

24. The method of any one of aspects 1-23, wherein the administering comprises intravenous administering.

25. The method of any one of aspects 1-23, wherein the administering comprises intrathecal administering.

26. A recombinant Adeno-associated viral (rAAV) vector comprising an AAV inverted terminal repeat, a promoter/enhancer, a nucleic acid sequence encoding sphingosine-1-phosphate lyase (SPL), and an AAV inverted terminal repeat.

27. The rAAV vector of aspect 26, wherein the SPL is a human SPL.

28. The rAAV vector of aspect 26 or 27, wherein the rAAV vector is AAV-PHP.B, AAV-PHP.eB, AAV-PHP.S or AAV-Anc80.

29. The rAAV vector of aspect 26 or 27, wherein the rAAV vector is AAV-9 vector.

30. The rAAV vector of any one of aspects 26-29, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin-Globin (CAG) Hybrid Promoter, or albumin promoter.

31. A recombinant adeno-associated viral (rAAV) virion comprising a nucleic acid encoding sphingosine-1-phosphate lyase (SPL).

32. The rAAV virion of aspect 31, comprising a rAAV vector comprising an AAV inverted terminal repeat, a promoter/enhancer, the nucleic acid sequence encoding sphingosine-1-phosphate lyase (SPL), and an AAV inverted terminal repeat.

33. The rAAV virion of aspect 31 or 32, wherein the SPL is a human SPL.

34. The rAAV virion of any one of aspects 31-33, wherein the rAAV virion comprises AAV serotype 9 capsid proteins.

35. The rAAV virion of any one of aspects 31-33, wherein the rAAV virion comprises AAV-PHP.B, AAV-PHP.eB, AAV-PHP.S or AAV-Anc80 capsids.

36. The rAAV vector of any one of aspects 31-35, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin-Globin (CAG) Hybrid Promoter, or albumin promoter.

37. A composition comprising the rAAV vector of any one of aspects 26-30 or the rAAV virion of any one of aspects 31-36 and a pharmaceutically acceptable excipient.

38. The rAAV vector of any one of aspects 26-30, the rAAV virion of any one of aspects 31-36, or the composition of aspect 37, for use in a method for a) treating SPL insufficiency syndrome (SPLIS) in a subject; b) preventing development of SPLIS in a subject; or c) reducing circulating sphingosine-1-phosphate (S1P) level in a subject, the method comprising administering the rAAV vector, the rAAV virion, or the composition to the subject wherein administering results in expression of SPL in the subject.

39. The rAAV vector, the rAAV virion, or the composition for use according to aspect 38, wherein the method comprises a) treating SPL insufficiency syndrome (SPLIS) in a subject.

40. The rAAV vector, the rAAV virion, or the composition for use according to aspect 39, wherein the subject has an increased level of plasma sphingosine-1-phosphate (S1P) and the administering results in at least a 5% reduction in plasma sphingosine-1-phosphate (S1P) in the subject.

41. The rAAV vector, the rAAV virion, or the composition for use according to aspect 39 or 40, wherein the subject has an increased level of C14 and 16 ceramides in the liver and the administering results in at least a 5% reduction in C14 and 16 ceramides in liver of the subject.

42. The rAAV vector, the rAAV virion, or the composition for use according to of any one of aspects 39-41, wherein the subject has hypoalbuminemia and the administering results in a reduction in hypoalbuminemia in the subject.

43. The rAAV vector, the rAAV virion, or the composition for use according to any one of aspects 39-42, wherein the subject has albuminuria and the administering results in a reduction in albuminuria in the subject.

44. The rAAV vector, the rAAV virion, or the composition for use according to any one of aspects 39-43, wherein the expression of SPL in the subject after the administering is at least 10% of the normal SPL level.

45. The rAAV vector, the rAAV virion, or the composition for use according to any one of aspects 39-44, wherein the subject has lymphopenia and is diagnosed as having SPLIS based on sequencing of the SGPL1 gene.

46. The rAAV vector, the rAAV virion, or the composition for use according to aspect 45, wherein the subject is a newborn.

47. The rAAV vector, the rAAV virion, or the composition for use according to aspect 38, wherein the method comprises b) preventing development of SPLIS in a subject.

48. The rAAV vector, the rAAV virion, or the composition for use according to aspect 47, wherein the subject has an inactivating mutation in SGPL1 gene.

49. The rAAV vector, the rAAV virion, or the composition for use according to aspect 47 or 48, wherein the subject is a fetus in utero, a newborn, an infant, a toddler, or a child.

50. The rAAV vector, the rAAV virion, or the composition for use according to any one of aspects 47-49, wherein the expression of SPL in the subject after the administering is at least 10% of the normal SPL level.

51. The rAAV vector, the rAAV virion, or the composition for use according to aspect 38, wherein the method comprises c) reducing circulating sphingosine-1-phosphate (S1P) level in a subject.

52. The rAAV vector, the rAAV virion, or the composition for use according to aspect 51, wherein the subject has or is at risk for developing SPL insufficiency syndrome (SPLIS).

53. The rAAV vector, the rAAV virion, or the composition for use according to aspect 51, wherein the subject has inflammation.

54. The rAAV vector, the rAAV virion, or the composition for use according to aspect 51, wherein the subject has cancer.

55. The rAAV vector, the rAAV virion, or the composition for use according to aspect 51, wherein the subject has inflammatory bowel disease.

56. The rAAV vector, the rAAV virion, or the composition for use according to aspect 51, wherein the subject has kidney disease.

57. The method of any one of aspects 1-23 or the rAAV vector, the rAAV virion, or the composition for use according to any one of aspects 97-114, wherein the therapeutically effective dose of the rAAV virion comprises a dose of about 10¹¹, 5×10¹¹, 10¹², 5×10¹², 10¹³, or 5×10¹³ viral genomes (vg).

58. A pharmaceutical composition comprising: the rAAV virion of any one of aspects 31-36 and a pharmaceutically acceptable diluent, carrier or excipient, wherein the composition comprises about 10¹¹, 5×10¹¹, 10¹², 5×10¹², 10¹³, or 5×10¹³ viral genomes (vg) of the rAAV virion.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Example 1: SPLIS Features

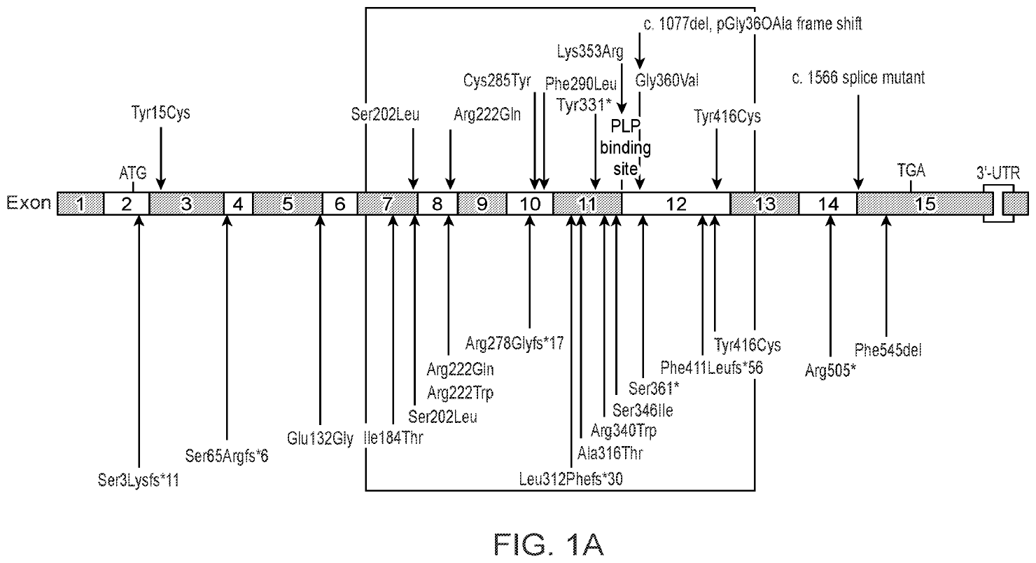

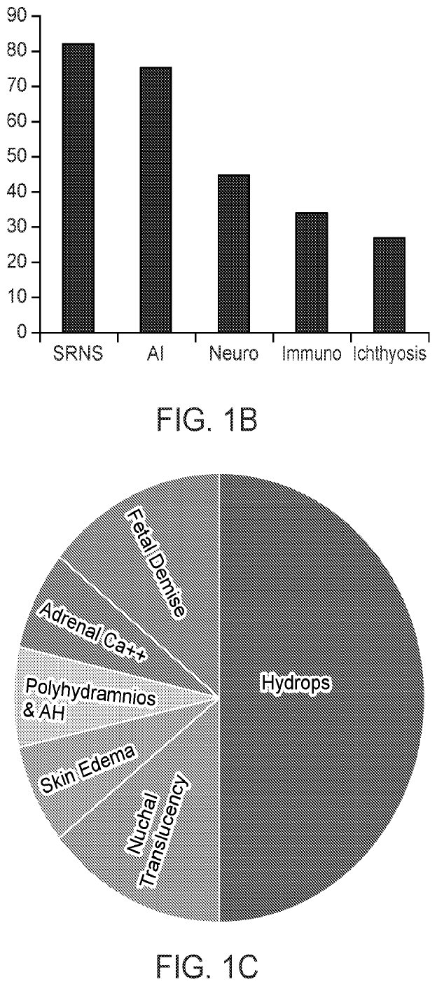

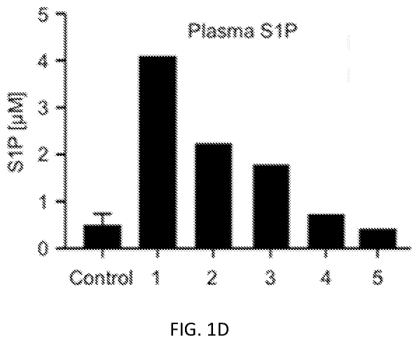

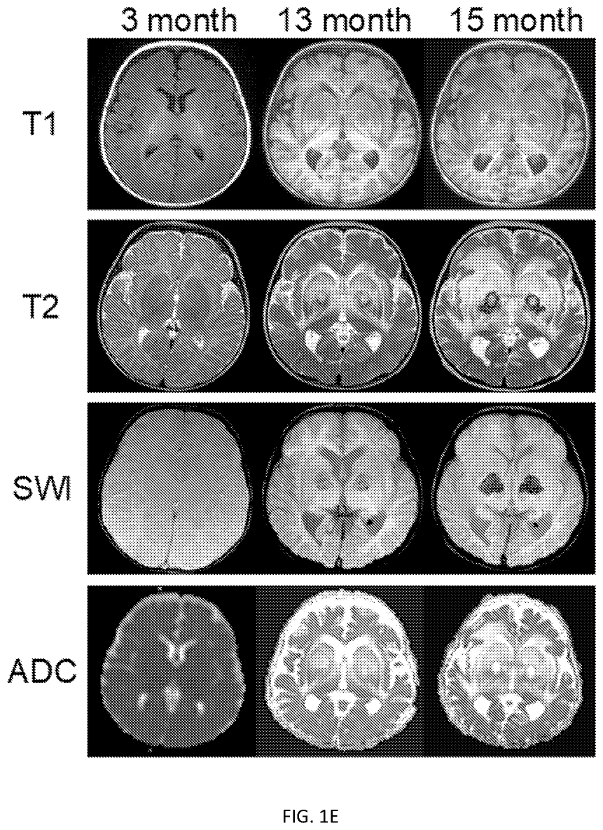

45 cases of SPLIS have been identified worldwide since publication of the first cohorts in February 2017. These include a majority of patients who lived at least 1 month and received a genetic diagnosis via whole exon or genome sequencing of index case and parents (FIG. 1A), and a handful of cases of fetal or perinatal death that were siblings of genetically confirmed index cases. The total SPLIS experience includes 37 reported cases plus 8 in our new case series. Of these, 4 were fetal losses, 17 are deceased and 24 living. The severity of SPLIS is wide ranging; most develop nephrotic syndrome and adrenal insufficiency (AI) (FIG. 1B). Patients with similar mutations show consistent phenotypes and severity. However, the natural history of the disease and the relationship between genetic mutation and clinical phenotype remain poorly understood. A variety of prenatal abnormalities have been observed (FIG. 1C). Two SPLIS patients were identified by low or absent T cell receptor excision circles (TREC) on newborn screening for severe combined immunodeficiency (Table 1). These findings indicate that SPLIS can potentially be identified by newborn screening.

TABLE 1
Immunological Findings in 7 SPLIS Patients

| | Leukocyte | | | Tcell | B cell | NK | IgG | Response to |
|---|---|---|---|---|---|---|---|---|
| Case | count | ALC | TRECs | counts | counts | counts | levels | Vaccines |
| 1 | 6.7 × 10⁶/L | 0.9 × 10⁶/L | ND | ND | ND | ND | ND | ND |
| 2 | 11.3 × 10⁶/L | 0.7 × 10⁶/L | ND | Low; CD3 and CD8 | | | | Protective |
| | | | | low | | | | |
| 3 | 4 × 10⁶/L | 1.0 × 10⁶/L | ND | Low; CD4 and CD8 | normal | low | Low IgG | Protective |
| | | | | low | | | and IgA | |
| 4 | 4 × 10⁶/L | 0.3 × 10⁶/L | ND | Low; Both | low | | Low | |
| | | | | CD4 and CD8 low | | | | |
| 5 | 7.5 × 10⁶/L | 0.4 × 10⁶/L | absent | Low; Distorted | low | low | ND | protective |
| | | | | distribution of naïve- | | | | |
| | | | | to-memory T cells | | | | |
| 6 | 13.7 × 10⁶/L | 7.1 × 10⁶/L | ND | ND | ND | ND | ND | ND |
| 7 | NA | NA | Very low | absolute CD3 T-cell | normal | normal | low | ND |
| | | | | 85 cells/μL | | | | |

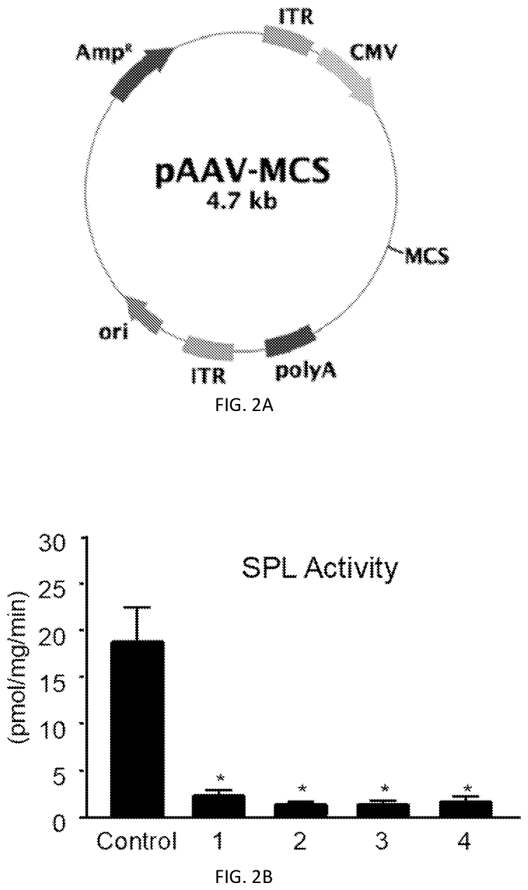

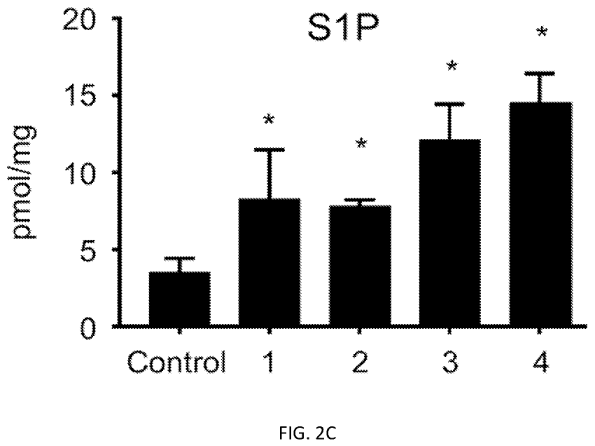

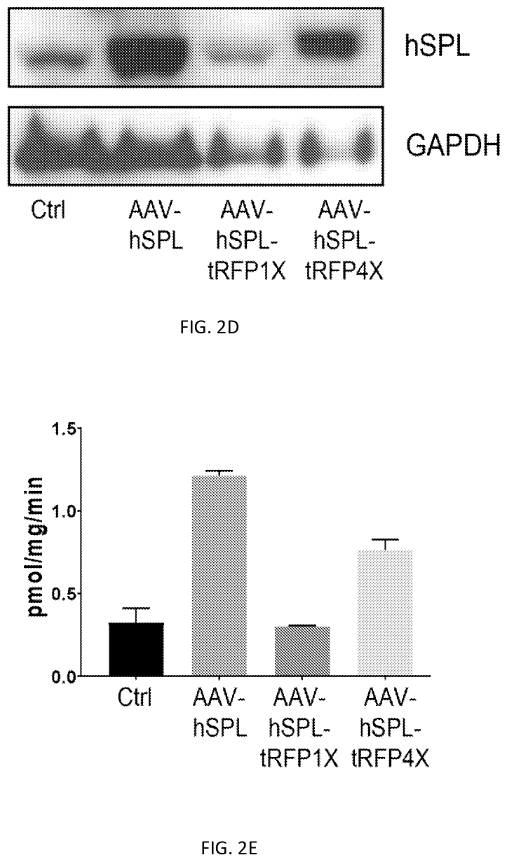

TERT-transformed skin fibroblast cultures from 4 SPLIS patients were developed and demonstrated lack of SPL activity and accumulation of S1P in all 4 SPLIS-derived cell lines (FIG. 2B,C). Similarly, most SPLIS patient plasma exhibited high levels of S1P compared to age/gender-matched controls (FIG. 1D). About half of SPLIS patients exhibit neurological involvement including sensorineural deafness, ptosis, paralysis with contractures, hypotonia, seizures, developmental delay or regression. Skin involvement is seen macroscopically as acanthosis/ichthyosis, and thickening of the corneal layer is observed on skin pathology. Nephrotic syndrome, and in particular SRNS with FSGS pathology is present in the majority of SPLIS patients. See J Lipid Res. 2019; 60(3):456-63.

MRI findings have identified callosal dysgenesis in two patients (not shown) and progressive changes associated with atrophy and basal ganglia involvement in three patients (example in FIG. 1E).

Immunological studies have not been conducted consistently in all of the known SPLIS patients. Hematological/immunological findings in our series suggests that anemia (not shown) and lymphopenia (Table 1) are present in most SPLIS patients. Deep profiling of lymphocyte subsets by CyTOF in one patient revealed reduced percentages and absolute levels (data not shown) of T, B and NKT lymphocytes. These findings are consistent with the high levels of S1P in most SPLIS patient plasma (FIG. 1D).

FIG. 1A. The SPL open reading frame encoded by SGPL1 is shown, with exons 1-15 indicated. The vitamin B6/PLP binding homology domain is shown by a box. Mutations found are shown in the schematic.

FIG. 1B. Major SPLIS features shown by percentage of patients. Steroid-resistant nephrotic syndrome (SRNS) is most frequent, followed by adrenal insufficiency and/or calcifications (AI), central or peripheral nervous system involvement (Neuro), immunodeficiency (Immuno) and ichthyosis. Immunodeficiency is likely to be underestimated due to reporting error.

FIG. 1C. Prenatal diagnoses associated with SPLIS (14 of 44 cases). AH, adrenal hemorrhage. Ca++, calcification.

FIG. 1D. Plasma S1P levels in five SPLIS patients. Plasma samples from SPLIS patients (1-5) and age- and gender-matched controls (N=7). Plasma (100 μL) was spiked with 10 μL internal standard mix (Avanti Polar Lipids) and extracted as described in Suh et al (2019). S1P was quantified using Agilent 1290 HPLC coupled with Agilent 6490 triple quadrupole mass spectrometer. Sphingolipid metabolites were resolved on a Zorbax RRHD Eclipse Plus C18 column. Conditions are described in Suh et al (2019) IBD Journal.

FIG. 1E. MRI findings shown by age and pulse sequence in a single SPLIS patient. Axial MRI T1, T2, and susceptibility weighted imaging (SWI) and diffusion weighted imaging as calculated absolute diffusion coefficient (ADC) of a SPLIS patient shows a variety of lesions consistent with edema, gliosis, microhemorrhage and indicative of progressive CNS involvement.

### Example 2: Generation of an AAV-SPL Expression System

Human WT SGPL1 cDNA or a self-cleaving system for coexpressing red fluorescence protein (RFP) and hSPL (hSPLtRFP) (Cold Spring Harb Protoc. 2012; 2012(2):199-204) were cloned in the multiple cloning site of pAAV-MCS, an AAV vector that contains the CMV promoter/enhancer and AAV2 inverted terminal repeats (ITR) (FIG. 2A). The resulting constructs were packaged in capsid AAV9. Viral particles were produced in an adenovirus-free system. AAV9 was selected for in vivo studies, based on its broad tropism including brain, adrenal gland and kidney—all of which are involved in SPLIS—as well as liver, a major site of metabolism of blood sphingolipids. A construct expressing an SPL protein that harbors a missense mutation at the cofactor binding lysine (AAV-SPL^{K353L}) shown previously to completely eliminate enzyme function was generated to serve as a biochemical control. AAV-SPL and AAV-SPL^{K353L} were produced at large scale yielding a solution with a concentration of 3.5×10³ vector genomes (vg) per mL.

TERT-transformed skin fibroblast cultures developed from 4 SPLIS patients demonstrate lack of SPL activity and accumulation of S1P in all 4 SPLIS-derived cell lines (FIG. 2B,C). Infection of SPLIS fibroblasts with AAV-hSPL or a 4-fold higher dose of AAV-hSPLtRFP increased SPL expression (FIG. 2D) and activity well above endogenous levels (FIG. 2E). Based on these results, AAV-hSPL was produced at larger scale for in vivo testing. AAV titer was quantified by Taqman qPCR of ITR primers, and probe was determined to be 3.5×10¹⁰ vector genomes (vg) per μL.

FIG. 2A. Schematic of an AAV vector. The pAAV-MCS vector. Human SGPL1 cDNA (hSPL) and hSPLtRFP were cloned into multiple cloning site (MCS) of pAAV-MCS (Cell Biolabs, Inc.) at EcoRI/Xho1 sites. Inserts were confirmed by DNA sequence analysis. This hSPL construct is labeled as “AAV-hSPL”.

FIGS. 2B,C show lack of SPL activity (FIG. 2B) and accumulation of S1P (FIG. 2C) in all 4 SPLIS-derived cell lines.

FIGS. 2D,E show that infection of SPLIS fibroblasts with AAV-hSPL or a 4-fold higher dose of AAV-hSPLtRFP increased SPL expression (FIG. 2D) and activity well above endogenous levels (FIG. 2E). *p<0.05.

Sequence of pAAV-CMV-hSPL-T2A-tRFP is set forth
in SEQ ID NO: 4:
(SEQ ID NO: 4)
CCTGCAGGCAGCTGCGCGCTCGCTCGCTCACTGAGG
CCGCCCGGGCAAAGCCCGGGCGTCGGGCGACCTTTG
GTCGCCCGGCCTCAGTGAGCGAGCGAGCGCGCAGAG
AGGGAGTGGCCAACTCCATCACTAGGGGTTCCTGCG
GCCGCACGCGTGGAGCTAGTTATTAATAGTAATCAA
TTACGGGGTCATTAGTTCATAGCCCATATATGGAGT
TCCGCGTTACATAACTTACGGTAAATGGCCCGCCTG
GCTGACCGCCCAACGACCCCCGCCCATTGACGTCAA
TAATGACGTATGTTCCCATAGTAACGTCAATAGGGA
CTTTCCATTGACGTCAATGGGTGGAGTATTTACGGT
AAACTGCCCACTTGGCAGTACATCAAGTGTATCATA
TGCCAAGTACGCCCCCTATTGACGTCAATGACGGTA
AATGGCCCGCCTGGCATTATGCCCAGTACATGACCT
TATGGGACTTTCCTACTTGGCAGTACATCTACGTAT
TAGTCATCGCTATTACCATGGTGATGCGGTTTTGGC
AGTACATCAATGGGCGTGGATAGCGGTTTGACTCAC
GGGGATTTCCAAGTCTCCACCCCATTGACGTCAATG
GGAGTTTGTTTTGCACCAAAATCAACGGGACTTTCC
AAAATGTCGTAACAACTCCGCCCCATTGACGCAAAT
GGGCGGTAGGCGTGTACGGTGGGAGGTCTATATAAG
CAGAGCTCGTTTAGTGAACCGTCAGATCGCCTGGAG
ACGCCATCCACGCTGTTTTGACCTCCATAGAAGACA
CCGGGACCGATCCAGCCTCCGCGGATTCGAATCCCG
GCCGGGAACGGTGCATTGGAACGCGGATTCCCCGTG
CCAAGAGTGACGTAAGTACCGCCTATAGAGTCTATA
GGCCCACAAAAAATGCTTTCTTCTTTTAATATACTT
TTTTGTTTATCTTATTTCTAATACTTTCCCTAATCT
CTTTCTTTCAGGGCAATAATGATACAATGTATCATG
CCTCTTTGCACCATTCTAAAGAATAACAGTGATAAT
TTCTGGGTTAAGGCAATAGCAATATTTCTGCATATA
AATATTTCTGCATATAAATTGTAACTGATGTAAGAG
GTTTCATATTGCTAATAGCAGCTACAATCCAGCTAC
CATTCTGCTTTTATTTTATGGTTGGGATAAGGCTGG
ATTATTCTGAGTCCAAGCTAGGCCCTTTTGCTAATC
ATGTTCATACCTCTTATCTTCCTCCCACAGCTCCTG
GGCAACGTGCTGGTCTGTGTGCTGGCCCATCACTTT
GGCAAAGAATTGGGATTCGAACATCGATTGAATTCG
CCACCATGCCTAGCACAGACCTTCTGATGTTGAAGG
CCTTTGAGCCCTACTTAGAGATTTTGGAAGTATACT
CCACAAAAGCCAAGAATTATGTAAATGGACATTGCA
CCAAGTATGAGCCCTGGCAGCTAATTGCATGGAGTG
TCGTGTGGACCCTGCTGATAGTCTGGGGATATGAGT
TTGTCTTCCAGCCAGAGAGTTTATGGTCAAGGTTTA
AAAAGAAATGTTTTAAGCTCACCAGGAAGATGCCCA
TTATTGGTCGTAAGATTCAAGACAAGTTGAACAAGA
CCAAGGATGATATTAGCAAGAACATGTCATTCCTGA
AAGTGGACAAAGAGTATGTGAAAGCTTTACCCTCCC
AGGGTCTGAGCTCATCTGCTGTTTTGGAGAAACTTA
AGGAGTACAGCTCTATGGACGCCTTCTGGCAAGAGG
GGAGAGCCTCTGGAACAGTGTACAGTGGGGAGGAGA
AGCTCACTGAGCTCCTTGTGAAGGCTTATGGAGATT
TTGCATGGAGTAACCCCCTGCATCCAGATATCTTCC
CAGGACTACGCAAGATAGAGGCAGAAATTGTGAGGA
TAGCTTGTTCCCTGTTCAATGGGGGACCAGATTCGT
GTGGATGTGTGACTTCTGGGGGAACAGAAAGCATAC
TGATGGCCTGCAAAGCATATCGGGATCTGGCCTTTG
AGAAGGGGATCAAAACTCCAGAAATTGTGGCTCCCC
AAAGTGCCCATGCTGCATTTAACAAAGCAGCCAGTT
ACTTTGGGATGAAGATTGTGCGGGTCCCATTGACGA
AGATGATGGAGGTGGATGTGCGGGCAATGAGAAGAG
CTATCTCCAGGAACACTGCCATGCTCGTCTGTTCTA
CCCCACAGTTTCCTCATGGTGTAATAGATCCTGTCC
CTGAAGTGGCCAAGCTGGCTGTCAAATACAAAATAC
CCCTTCATGTCGACGCTTGTCTGGGAGGCTTCCTCA
TCGTCTTTATGGAGAAAGCAGGATACCCACTGGAGC
ACCCATTTGATTTCCGGGTGAAAGGTGTAACCAGCA
TTTCAGCTGACACCCATAAGTATGGCTATGCCCCAA
AAGGCTCATCATTGGTGTTGTATAGTGACAAGAAGT
ACAGGAACTATCAGTTCTTCGTCGATACAGATTGGC
AGGGTGGCATCTATGCTTCCCCAACCATCGCAGGCT
CACGGCCTGGTGGCATTAGCGCAGCCTGTTGGGCTG
CCTTGATGCACTTCGGTGAGAACGGCTATGTTGAAG
CTACCAAACAGATCATCAAAACTGCTCGCTTCCTCA
AGTCAGAACTGGAAAATATCAAAGGCATCTTTGTTT
TTGGGAATCCCCAATTGTCAGTCATTGCTCTGGGAT
CCCGTGATTTTGACATCTACCGACTATCAAACCTGA
TGACTGCTAAGGGGTGGAACTTGAACCAGTTGCAGT
TCCCACCCAGTATTCATTTCTGCATCACATTACTAC
ACGCCCGGAAACGAGTAGCTATACAATTCCTAAAGG
ACATTCGAGAATCTGTCACTCAAATCATGAAGAATC
CTAAAGCGAAGACCACAGGAATGGGTGCCATCTATG
GCATGGCCCAGACAACTGTTGACAGGAATATGGTTG
CAGAATTGTCCTCAGTCTTCTTGGACAGCTTGTACA
GCACCGACACTGTCACCCAGGGCAGCCAGATGAATG
GTTCTCCAAAACCACACGAGGGCAGAGGAAGTCTTC
TAACATGCGGTGACGTGGAGGAGAATCCCGGCCCTA
TGGTGTCTAAGGGCGAAGAGCTGATTAAGGAGAACA
TGCACATGAAGCTGTACATGGAGGGCACCGTGAACA
ACCACCACTTCAAGTGCACATCCGAGGGCGAAGGCA
AGCCCTACGAGGGCACCCAGACCATGAGAATCAAGG
TGGTCGAGGGCGGCCCTCTCCCCTTCGCCTTCGACA
TCCTGGCTACCAGCTTCATGTACGGCAGCAGAACCT
TCATCAACCACACCCAGGGCATCCCCGACTTCTTTA
AGCAGTCCTTCCCTGAGGGCTTCACATGGGAGAGAG
TCACCACATACGAAGACGGGGGCGTGCTGACCGCTA
CCCAGGACACCAGCCTCCAGGACGGCTGCCTCATCT
ACAACGTCAAGATCAGAGGGGTGAACTTCCCATCCA
ACGGCCCTGTGATGCAGAAGAAAACACTCGGCTGGG
AGGCCAACACCGAGATGCTGTACCCCGCTGACGGCG
GCCTGGAAGGCAGAAGCGACATGGCCCTGAAGCTCG
TGGGCGGGGGCCACCTGATCTGCAACTTCAAGACCA
CATACAGATCCAAGAAACCCGCTAAGAACCTCAAGA
TGCCCGGCGTCTACTATGTGGACCACAGACTGGAAA
GAATCAAGGAGGCCGACAAAGAGACCTACGTCGAGC
AGCACGAGGTGGCTGTGGCCAGATACTGCGACCTCC
CTAGCAAACTGGGGCACAAACTTAATTGACTCGAGA
GATCTACGGGTGGCATCCCTGTGACCCCTCCCCAGT
GCCTCTCCTGGCCCTGGAAGTTGCCACTCCAGTGCC
CACCAGCCTTGTCCTAATAAAATTAAGTTGCATCAT
TTTGTCTGACTAGGTGTCCTTCTATAATATTATGGG
GTGGAGGGGGGTGGTATGGAGCAAGGGGCAAGTTGG
GAAGACAACCTGTAGGGCCTGCGGGGTCTATTGGGA
ACCAAGCTGGAGTGCAGTGGCACAATCTTGGCTCAC
TGCAATCTCCGCCTCCTGGGTTCAAGCGATTCTCCT
GCCTCAGCCTCCCGAGTTGTTGGGATTCCAGGCATG
CATGACCAGGCTCAGCTAATTTTTGTTTTTTTGGTA
GAGACGGGGTTTCACCATATTGGCCAGGCTGGTCTC
CAACTCCTAATCTCAGGTGATCTACCCACCTTGGCC
TCCCAAATTGCTGGGATTACAGGCGTGAACCACTGC
TCCCTTCCCTGTCCTTCTGATTTTGTAGGTAACCAC
GTGCGGACCGAGCGGCCGCAGGAACCCCTAGTGATG
GAGTTGGCCACTCCCTCTCTGCGCGCTCGCTCGCTC
ACTGAGGCCGGGCGACCAAAGGTCGCCCGACGCCCG
GGCTTTGCCCGGGCGGCCTCAGTGAGCGAGCGAGCG
CGCAGCTGCCTGCAGGGGCGCCTGATGCGGTATTTT
CTCCTTACGCATCTGTGCGGTATTTCACACCGCATA
CGTCAAAGCAACCATAGTACGCGCCCTGTAGCGGCG
CATTAAGCGCGGCGGGTGTGGTGGTTACGCGCAGCG
TGACCGCTACACTTGCCAGCGCCCTAGCGCCCGCTC
CTTTCGCTTTCTTCCCTTCCTTTCTCGCCACGTTCG
CCGGCTTTCCCCGTCAAGCTCTAAATCGGGGGCTCC
CTTTAGGGTTCCGATTTAGTGCTTTACGGCACCTCG
ACCCCAAAAAACTTGATTTGGGTGATGGTTCACGTA
GTGGGCCATCGCCCTGATAGACGGTTTTTCGCCCTT
TGACGTTGGAGTCCACGTTCTTTAATAGTGGACTCT
TGTTCCAAACTGGAACAACACTCAACCCTATCTCGG
GCTATTCTTTTGATTTATAAGGGATTTTGCCGATTT
CGGCCTATTGGTTAAAAAATGAGCTGATTTAACAAA
AATTTAACGCGAATTTTAACAAAATATTAACGTTTA
CAATTTTATGGTGCACTCTCAGTACAATCTGCTCTG
ATGCCGCATAGTTAAGCCAGCCCCGACACCCGCCAA
CACCCGCTGACGCGCCCTGACGGGCTTGTCTGCTCC
CGGCATCCGCTTACAGACAAGCTGTGACCGTCTCCG
GGAGCTGCATGTGTCAGAGGTTTTCACCGTCATCAC
CGAAACGCGCGAGACGAAAGGGCCTCGTGATACGCC
TATTTTTATAGGTTAATGTCATGATAATAATGGTTT
CTTAGACGTCAGGTGGCACTTTTCGGGGAAATGTGC
GCGGAACCCCTATTTGTTTATTTTTCTAAATACATT
CAAATATGTATCCGCTCATGAGACAATAACCCTGAT
AAATGCTTCAATAATATTGAAAAAGGAAGAGTATGA
GTATTCAACATTTCCGTGTCGCCCTTATTCCCTTTT
TTGCGGCATTTTGCCTTCCTGTTTTTGCTCACCCAG
AAACGCTGGTGAAAGTAAAAGATGCTGAAGATCAGT
TGGGTGCACGAGTGGGTTACATCGAACTGGATCTCA
ACAGCGGTAAGATCCTTGAGAGTTTTCGCCCCGAAG
AACGTTTTCCAATGATGAGCACTTTTAAAGTTCTGC
TATGTGGCGCGGTATTATCCCGTATTGACGCCGGGC
AAGAGCAACTCGGTCGCCGCATACACTATTCTCAGA
ATGACTTGGTTGAGTACTCACCAGTCACAGAAAAGC
ATCTTACGGATGGCATGACAGTAAGAGAATTATGCA
GTGCTGCCATAACCATGAGTGATAACACTGCGGCCA
ACTTACTTCTGACAACGATCGGAGGACCGAAGGAGC
TAACCGCTTTTTTGCACAACATGGGGGATCATGTAA
CTCGCCTTGATCGTTGGGAACCGGAGCTGAATGAAG
CCATACCAAACGACGAGCGTGACACCACGATGCCTG
TAGCAATGGCAACAACGTTGCGCAAACTATTAACTG
GCGAACTACTTACTCTAGCTTCCCGGCAACAATTAA
TAGACTGGATGGAGGCGGATAAAGTTGCAGGACCAC
TTCTGCGCTCGGCCCTTCCGGCTGGCTGGTTTATTG
CTGATAAATCTGGAGCCGGTGAGCGTGGGTCTCGCG
GTATCATTGCAGCACTGGGGCCAGATGGTAAGCCCT
CCCGTATCGTAGTTATCTACACGACGGGGAGTCAGG
CAACTATGGATGAACGAAATAGACAGATCGCTGAGA
TAGGTGCCTCACTGATTAAGCATTGGTAACTGTCAG
ACCAAGTTTACTCATATATACTTTAGATTGATTTAA
AACTTCATTTTTAATTTAAAAGGATCTAGGTGAAGA
TCCTTTTTGATAATCTCATGACCAAAATCCCTTAAC
GTGAGTTTTCGTTCCACTGAGCGTCAGACCCCGTAG
AAAAGATCAAAGGATCTTCTTGAGATCCTTTTTTTC
TGCGCGTAATCTGCTGCTTGCAAACAAAAAAACCAC
CGCTACCAGCGGTGGTTTGTTTGCCGGATCAAGAGC
TACCAACTCTTTTTCCGAAGGTAACTGGCTTCAGCA
GAGCGCAGATACCAAATACTGTCCTTCTAGTGTAGC
CGTAGTTAGGCCACCACTTCAAGAACTCTGTAGCAC
CGCCTACATACCTCGCTCTGCTAATCCTGTTACCAG
TGGCTGCTGCCAGTGGCGATAAGTCGTGTCTTACCG
GGTTGGACTCAAGACGATAGTTACCGGATAAGGCGC
AGCGGTCGGGCTGAACGGGGGGTTCGTGCACACAGC
CCAGCTTGGAGCGAACGACCTACACCGAACTGAGAT
ACCTACAGCGTGAGCTATGAGAAAGCGCCACGCTTC
CCGAAGGGAGAAAGGCGGACAGGTATCCGGTAAGCG
GCAGGGTCGGAACAGGAGAGCGCACGAGGGAGCTTC
CAGGGGGAAACGCCTGGTATCTTTATAGTCCTGTCG
GGTTTCGCCACCTCTGACTTGAGCGTCGATTTTTGT
GATGCTCGTCAGGGGGGCGGAGCCTATGGAAAAACG
CCAGCAACGCGGCCTTTTTACGGTTCCTGGCCTTTT
GCTGGCCTTTTGCTCACATGT.
Sequence of pAAV-CMV-hSPL is set forth
in SEQ ID NO: 5:
(SEQ ID NO: 5)
CCTGCAGGCAGCTGCGCGCTCGCTCGCTCACTGAGG
CCGCCCGGGCAAAGCCCGGGCGTCGGGCGACCTTTG
GTCGCCCGGCCTCAGTGAGCGAGCGAGCGCGCAGAG
AGGGAGTGGCCAACTCCATCACTAGGGGTTCCTGCG
GCCGCACGCGTGGAGCTAGTTATTAATAGTAATCAA
TTACGGGGTCATTAGTTCATAGCCCATATATGGAGT
TCCGCGTTACATAACTTACGGTAAATGGCCCGCCTG
GCTGACCGCCCAACGACCCCCGCCCATTGACGTCAA
TAATGACGTATGTTCCCATAGTAACGTCAATAGGGA
CTTTCCATTGACGTCAATGGGTGGAGTATTTACGGT
AAACTGCCCACTTGGCAGTACATCAAGTGTATCATA
TGCCAAGTACGCCCCCTATTGACGTCAATGACGGTA
AATGGCCCGCCTGGCATTATGCCCAGTACATGACCT
TATGGGACTTTCCTACTTGGCAGTACATCTACGTAT
TAGTCATCGCTATTACCATGGTGATGCGGTTTTGGC
AGTACATCAATGGGCGTGGATAGCGGTTTGACTCAC
GGGGATTTCCAAGTCTCCACCCCATTGACGTCAATG
GGAGTTTGTTTTGCACCAAAATCAACGGGACTTTCC
AAAATGTCGTAACAACTCCGCCCCATTGACGCAAAT
GGGCGGTAGGCGTGTACGGTGGGAGGTCTATATAAG
CAGAGCTCGTTTAGTGAACCGTCAGATCGCCTGGAG
ACGCCATCCACGCTGTTTTGACCTCCATAGAAGACA
CCGGGACCGATCCAGCCTCCGCGGATTCGAATCCCG
GCCGGGAACGGTGCATTGGAACGCGGATTCCCCGTG
CCAAGAGTGACGTAAGTACCGCCTATAGAGTCTATA
GGCCCACAAAAAATGCTTTCTTCTTTTAATATACTT
TTTTGTTTATCTTATTTCTAATACTTTCCCTAATCT
CTTTCTTTCAGGGCAATAATGATACAATGTATCATG
CCTCTTTGCACCATTCTAAAGAATAACAGTGATAAT
TTCTGGGTTAAGGCAATAGCAATATTTCTGCATATA
AATATTTCTGCATATAAATTGTAACTGATGTAAGAG
GTTTCATATTGCTAATAGCAGCTACAATCCAGCTAC
CATTCTGCTTTTATTTTATGGTTGGGATAAGGCTGG
ATTATTCTGAGTCCAAGCTAGGCCCTTTTGCTAATC
ATGTTCATACCTCTTATCTTCCTCCCACAGCTCCTG
GGCAACGTGCTGGTCTGTGTGCTGGCCCATCACTTT
GGCAAAGAATTGGGATTCGAACATCGATTGAATTCG
CCACCATGCCTAGCACAGACCTTCTGATGTTGAAGG
CCTTTGAGCCCTACTTAGAGATTTTGGAAGTATACT
CCACAAAAGCCAAGAATTATGTAAATGGACATTGCA
CCAAGTATGAGCCCTGGCAGCTAATTGCATGGAGTG
TCGTGTGGACCCTGCTGATAGTCTGGGGATATGAGT
TTGTCTTCCAGCCAGAGAGTTTATGGTCAAGGTTTA
AAAAGAAATGTTTTAAGCTCACCAGGAAGATGCCCA
TTATTGGTCGTAAGATTCAAGACAAGTTGAACAAGA
CCAAGGATGATATTAGCAAGAACATGTCATTCCTGA
AAGTGGACAAAGAGTATGTGAAAGCTTTACCCTCCC
AGGGTCTGAGCTCATCTGCTGTTTTGGAGAAACTTA
AGGAGTACAGCTCTATGGACGCCTTCTGGCAAGAGG
GGAGAGCCTCTGGAACAGTGTACAGTGGGGAGGAGA
AGCTCACTGAGCTCCTTGTGAAGGCTTATGGAGATT
TTGCATGGAGTAACCCCCTGCATCCAGATATCTTCC
CAGGACTACGCAAGATAGAGGCAGAAATTGTGAGGA
TAGCTTGTTCCCTGTTCAATGGGGGACCAGATTCGT
GTGGATGTGTGACTTCTGGGGGAACAGAAAGCATAC
TGATGGCCTGCAAAGCATATCGGGATCTGGCCTTTG
AGAAGGGGATCAAAACTCCAGAAATTGTGGCTCCCC
AAAGTGCCCATGCTGCATTTAACAAAGCAGCCAGTT
ACTTTGGGATGAAGATTGTGCGGGTCCCATTGACGA
AGATGATGGAGGTGGATGTGCGGGCAATGAGAAGAG
CTATCTCCAGGAACACTGCCATGCTCGTCTGTTCTA
CCCCACAGTTTCCTCATGGTGTAATAGATCCTGTCC
CTGAAGTGGCCAAGCTGGCTGTCAAATACAAAATAC
CCCTTCATGTCGACGCTTGTCTGGGAGGCTTCCTCA
TCGTCTTTATGGAGAAAGCAGGATACCCACTGGAGC
ACCCATTTGATTTCCGGGTGAAAGGTGTAACCAGCA
TTTCAGCTGACACCCATAAGTATGGCTATGCCCCAA
AAGGCTCATCATTGGTGTTGTATAGTGACAAGAAGT
ACAGGAACTATCAGTTCTTCGTCGATACAGATTGGC
AGGGTGGCATCTATGCTTCCCCAACCATCGCAGGCT
CACGGCCTGGTGGCATTAGCGCAGCCTGTTGGGCTG
CCTTGATGCACTTCGGTGAGAACGGCTATGTTGAAG
CTACCAAACAGATCATCAAAACTGCTCGCTTCCTCA
AGTCAGAACTGGAAAATATCAAAGGCATCTTTGTTT
TTGGGAATCCCCAATTGTCAGTCATTGCTCTGGGAT
CCCGTGATTTTGACATCTACCGACTATCAAACCTGA
TGACTGCTAAGGGGTGGAACTTGAACCAGTTGCAGT
TCCCACCCAGTATTCATTTCTGCATCACATTACTAC
ACGCCCGGAAACGAGTAGCTATACAATTCCTAAAGG
ACATTCGAGAATCTGTCACTCAAATCATGAAGAATC
CTAAAGCGAAGACCACAGGAATGGGTGCCATCTATG
GCATGGCCCAGACAACTGTTGACAGGAATATGGTTG
CAGAATTGTCCTCAGTCTTCTTGGACAGCTTGTACA
GCACCGACACTGTCACCCAGGGCAGCCAGATGAATG
GTTCTCCAAAACCCCACTGACTCGAGAGATCTACGG
GTGGCATCCCTGTGACCCCTCCCCAGTGCCTCTCCT
GGCCCTGGAAGTTGCCACTCCAGTGCCCACCAGCCT
TGTCCTAATAAAATTAAGTTGCATCATTTTGTCTGA
CTAGGTGTCCTTCTATAATATTATGGGGTGGAGGGG
GGTGGTATGGAGCAAGGGGCAAGTTGGGAAGACAAC
CTGTAGGGCCTGCGGGGTCTATTGGGAACCAAGCTG
GAGTGCAGTGGCACAATCTTGGCTCACTGCAATCTC
CGCCTCCTGGGTTCAAGCGATTCTCCTGCCTCAGCC
TCCCGAGTTGTTGGGATTCCAGGCATGCATGACCAG
GCTCAGCTAATTTTTGTTTTTTTGGTAGAGACGGGG
TTTCACCATATTGGCCAGGCTGGTCTCCAACTCCTA
ATCTCAGGTGATCTACCCACCTTGGCCTCCCAAATT
GCTGGGATTACAGGCGTGAACCACTGCTCCCTTCCC
TGTCCTTCTGATTTTGTAGGTAACCACGTGCGGACC
GAGCGGCCGCAGGAACCCCTAGTGATGGAGTTGGCC
ACTCCCTCTCTGCGCGCTCGCTCGCTCACTGAGGCC
GGGCGACCAAAGGTCGCCCGACGCCCGGGCTTTGCC
CGGGCGGCCTCAGTGAGCGAGCGAGCGCGCAGCTGC
CTGCAGGGGCGCCTGATGCGGTATTTTCTCCTTACG
CATCTGTGCGGTATTTCACACCGCATACGTCAAAGC
AACCATAGTACGCGCCCTGTAGCGGCGCATTAAGCG
CGGCGGGTGTGGTGGTTACGCGCAGCGTGACCGCTA
CACTTGCCAGCGCCCTAGCGCCCGCTCCTTTCGCTT
TCTTCCCTTCCTTTCTCGCCACGTTCGCCGGCTTTC
CCCGTCAAGCTCTAAATCGGGGGCTCCCTTTAGGGT
TCCGATTTAGTGCTTTACGGCACCTCGACCCCAAAA
AACTTGATTTGGGTGATGGTTCACGTAGTGGGCCAT
CGCCCTGATAGACGGTTTTTCGCCCTTTGACGTTGG
AGTCCACGTTCTTTAATAGTGGACTCTTGTTCCAAA
CTGGAACAACACTCAACCCTATCTCGGGCTATTCTT
TTGATTTATAAGGGATTTTGCCGATTTCGGCCTATT
GGTTAAAAAATGAGCTGATTTAACAAAAATTTAACG
CGAATTTTAACAAAATATTAACGTTTACAATTTTAT
GGTGCACTCTCAGTACAATCTGCTCTGATGCCGCAT
AGTTAAGCCAGCCCCGACACCCGCCAACACCCGCTG
ACGCGCCCTGACGGGCTTGTCTGCTCCCGGCATCCG
CTTACAGACAAGCTGTGACCGTCTCCGGGAGCTGCA
TGTGTCAGAGGTTTTCACCGTCATCACCGAAACGCG
CGAGACGAAAGGGCCTCGTGATACGCCTATTTTTAT
AGGTTAATGTCATGATAATAATGGTTTCTTAGACGT
CAGGTGGCACTTTTCGGGGAAATGTGCGCGGAACCC
CTATTTGTTTATTTTTCTAAATACATTCAAATATGT
ATCCGCTCATGAGACAATAACCCTGATAAATGCTTC
AATAATATTGAAAAAGGAAGAGTATGAGTATTCAAC
ATTTCCGTGTCGCCCTTATTCCCTTTTTTGCGGCAT
TTTGCCTTCCTGTTTTTGCTCACCCAGAAACGCTGG
TGAAAGTAAAAGATGCTGAAGATCAGTTGGGTGCAC
GAGTGGGTTACATCGAACTGGATCTCAACAGCGGTA
AGATCCTTGAGAGTTTTCGCCCCGAAGAACGTTTTC
CAATGATGAGCACTTTTAAAGTTCTGCTATGTGGCG
CGGTATTATCCCGTATTGACGCCGGGCAAGAGCAAC
TCGGTCGCCGCATACACTATTCTCAGAATGACTTGG
TTGAGTACTCACCAGTCACAGAAAAGCATCTTACGG
ATGGCATGACAGTAAGAGAATTATGCAGTGCTGCCA
TAACCATGAGTGATAACACTGCGGCCAACTTACTTC
TGACAACGATCGGAGGACCGAAGGAGCTAACCGCTT
TTTTGCACAACATGGGGGATCATGTAACTCGCCTTG
ATCGTTGGGAACCGGAGCTGAATGAAGCCATACCAA
ACGACGAGCGTGACACCACGATGCCTGTAGCAATGG
CAACAACGTTGCGCAAACTATTAACTGGCGAACTAC
TTACTCTAGCTTCCCGGCAACAATTAATAGACTGGA
TGGAGGCGGATAAAGTTGCAGGACCACTTCTGCGCT
CGGCCCTTCCGGCTGGCTGGTTTATTGCTGATAAAT
CTGGAGCCGGTGAGCGTGGGTCTCGCGGTATCATTG
CAGCACTGGGGCCAGATGGTAAGCCCTCCCGTATCG
TAGTTATCTACACGACGGGGAGTCAGGCAACTATGG
ATGAACGAAATAGACAGATCGCTGAGATAGGTGCCT
CACTGATTAAGCATTGGTAACTGTCAGACCAAGTTT
ACTCATATATACTTTAGATTGATTTAAAACTTCATT
TTTAATTTAAAAGGATCTAGGTGAAGATCCTTTTTG
ATAATCTCATGACCAAAATCCCTTAACGTGAGTTTT
CGTTCCACTGAGCGTCAGACCCCGTAGAAAAGATCA
AAGGATCTTCTTGAGATCCTTTTTTTCTGCGCGTAA
TCTGCTGCTTGCAAACAAAAAAACCACCGCTACCAG
CGGTGGTTTGTTTGCCGGATCAAGAGCTACCAACTC
TTTTTCCGAAGGTAACTGGCTTCAGCAGAGCGCAGA
TACCAAATACTGTCCTTCTAGTGTAGCCGTAGTTAG
GCCACCACTTCAAGAACTCTGTAGCACCGCCTACAT
ACCTCGCTCTGCTAATCCTGTTACCAGTGGCTGCTG
CCAGTGGCGATAAGTCGTGTCTTACCGGGTTGGACT
CAAGACGATAGTTACCGGATAAGGCGCAGCGGTCGG
GCTGAACGGGGGGTTCGTGCACACAGCCCAGCTTGG
AGCGAACGACCTACACCGAACTGAGATACCTACAGC
GTGAGCTATGAGAAAGCGCCACGCTTCCCGAAGGGA
GAAAGGCGGACAGGTATCCGGTAAGCGGCAGGGTCG
GAACAGGAGAGCGCACGAGGGAGCTTCCAGGGGGAA
ACGCCTGGTATCTTTATAGTCCTGTCGGGTTTCGCC
ACCTCTGACTTGAGCGTCGATTTTTGTGATGCTCGT
CAGGGGGGCGGAGCCTATGGAAAAACGCCAGCAACG
CGGCCTTTTTACGGTTCCTGGCCTTTTGCTGGCCTT
TTGCTCACATGT.

### Example 3: Administration of AAV-HSPL to Newborn SPL Knockout (KO) Pups

To test the ability of AAV-hSPL to express functional SPL in vivo, it was delivered to newborn SPL KO pups. SPL KO mice are described in Nat Genet. 2007; 39(1):52-60. SPL KO mice are characterized by short lifespan, runting, anemia and infrequent defects of bone and kidney.

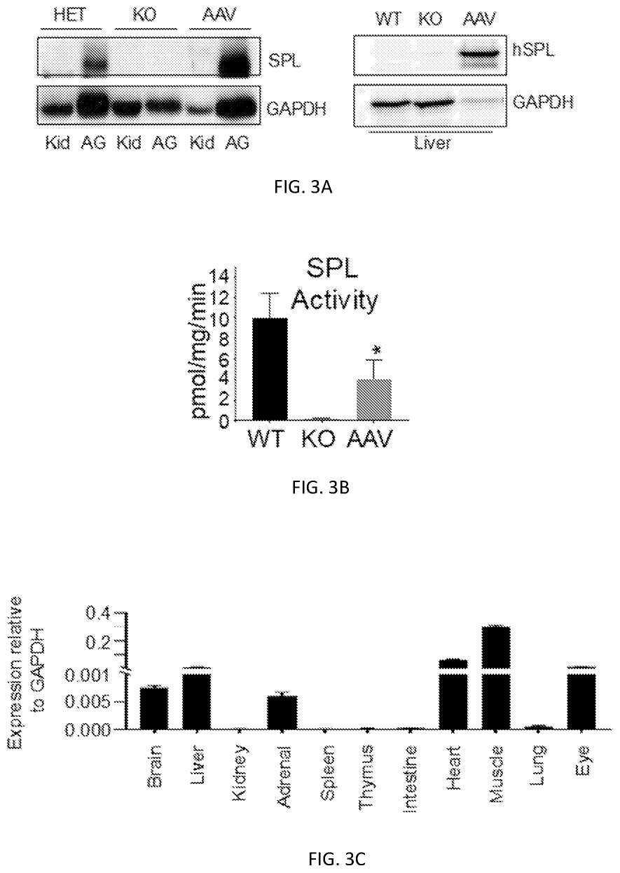

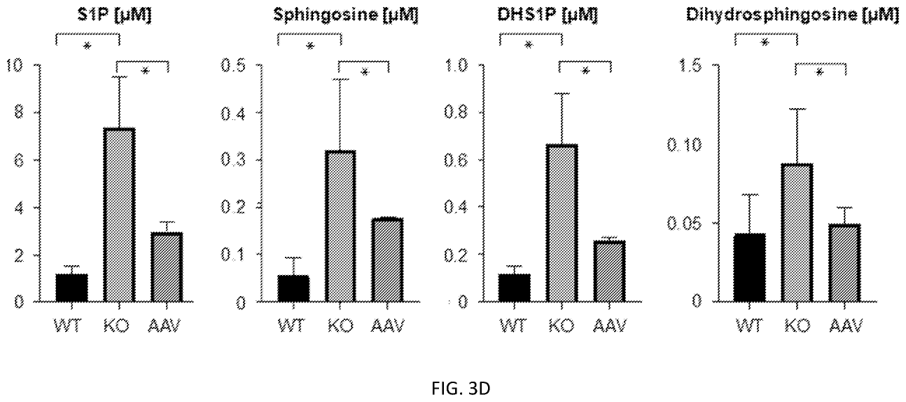

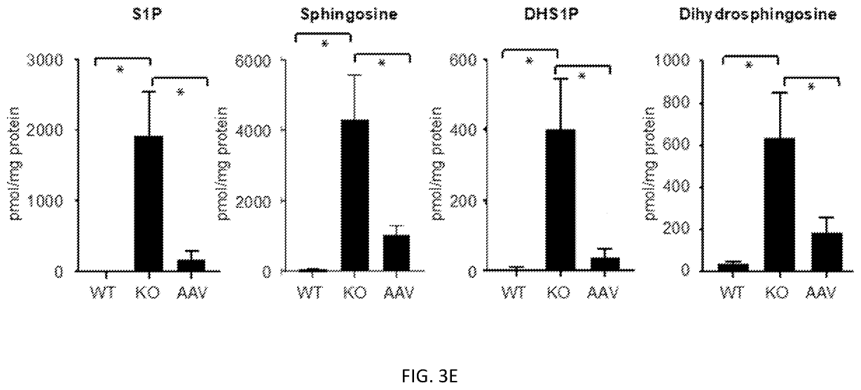

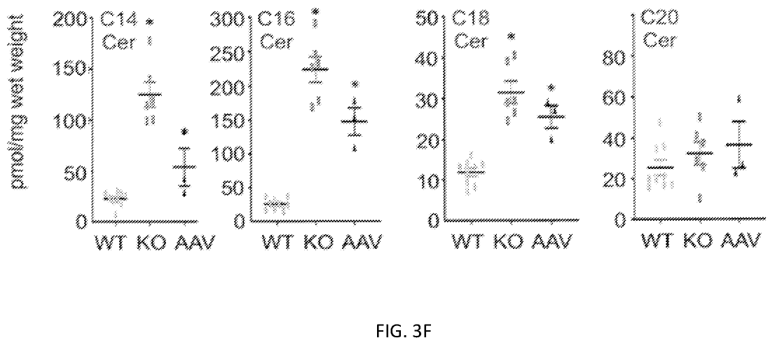

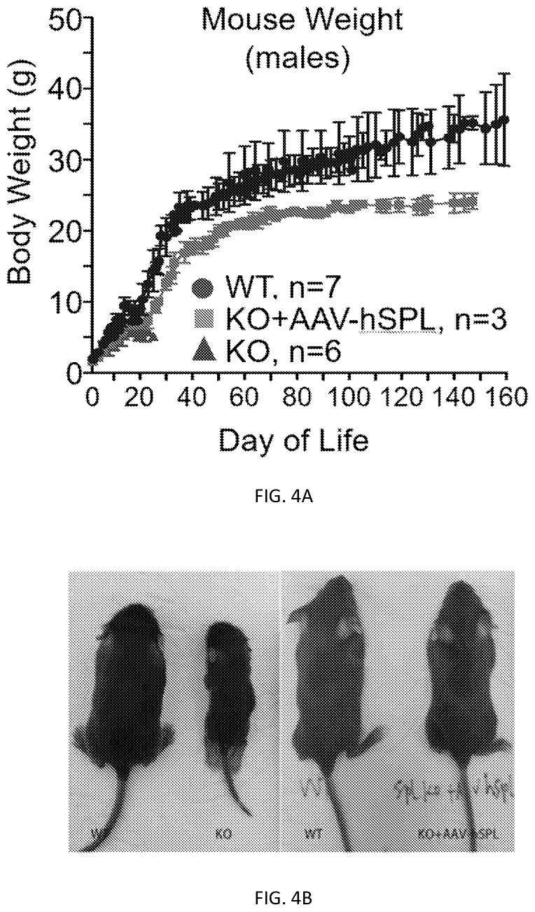

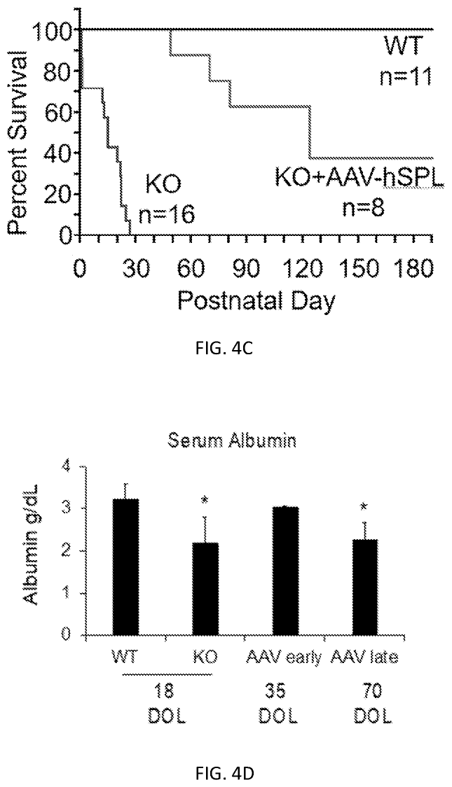

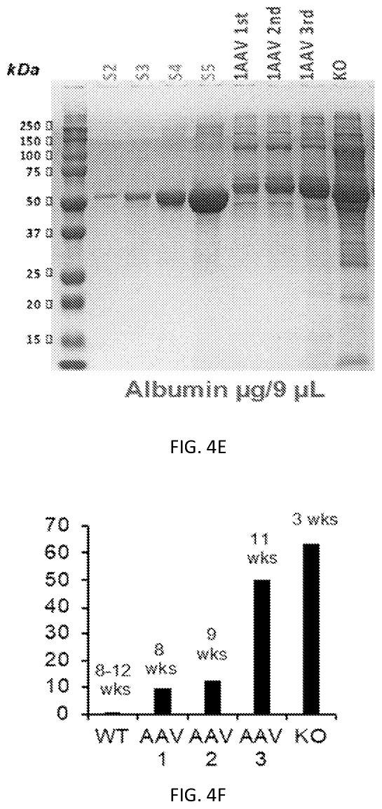

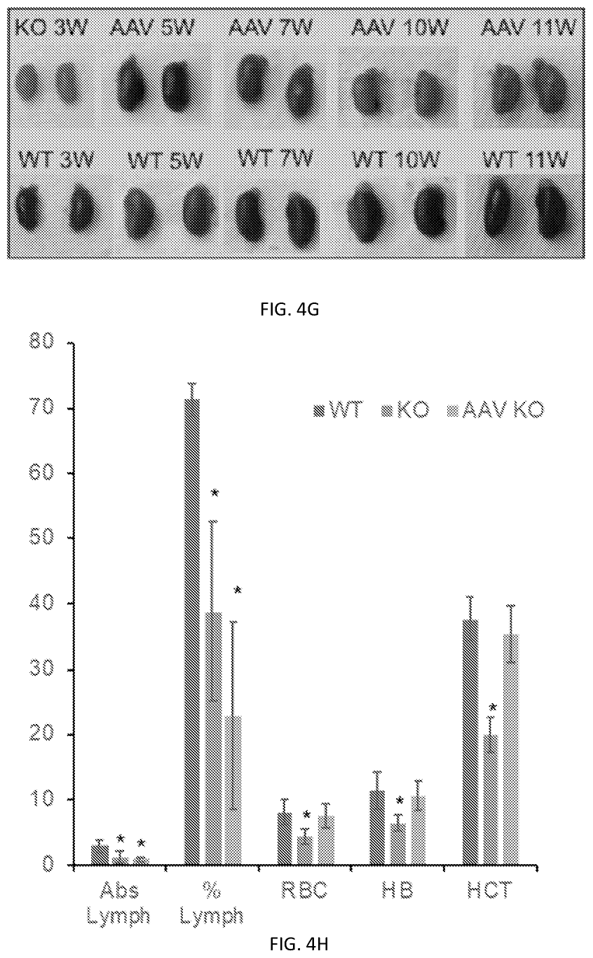

Heterozygous “het” SPL KO were crossed and the resulting pups were genotyped at day of life (DOL) 1 by toe biopsy. SPL KO pups were treated at 2-3 DOL with a single dose of 10-20 μL of a solution containing 3.5×10¹⁰ vg/μL by intravenous (IV) injection. At DOL 21, AAV-treated KO pups and age-matched controls were sacrificed and tissues collected. SPL expression was compared in homogenates of liver, kidney and adrenal gland by western blotting (WB) using either commercial human-SPL specific Ab or an anti-mSPL which detects both mSPL and hSPL. As shown in FIG. 3A, AAV-hSPL treated KO mice showed increased hSPL expression in liver and adrenal gland with a faint band in kidney compared to untreated KO. SPL activity levels in AAV-hSPL treated KO liver were ˜40% of WT control levels (FIG. 3B). Using qRT-PCR, hSPL expression was observed in different tissues of AAV-hSPL treated KO mice (FIG. 3C). Consistent with the increased SPL activity, high plasma S1P and other sphingolipids in KO mice were reduced in AAV-hSPL treated KO mice (FIG. 3D). High S1P and other sphingolipid levels in KO liver were reduced in AAV-hSPL treated KO liver (FIG. 3E). C14 and 16 ceramides were also elevated in the KO and reduced in treated liver, whereas ceramides C20 and above were not significantly different in WT, treated or untreated KO liver (FIG. 3F). To establish whether the partial normalization of sphingolipid levels observed correlated with improvement in the health status of SPL KO mice, AAV-hSPL was administered in a single dose to an additional 6 SPL KO pups at DOL 2-3. Pups were monitored for weight gain and were euthanized if the humane endpoint was reached. As shown in FIG. 4A-B, AAV-hSPL treated KO pups showed improvement in weight gain compared to untreated KO controls. Treated KO mouse weights were between 85-100% of gender- and age-matched WT and het controls at each age up to adulthood. Mean survival increased from 18 days in untreated KO controls to 80 days in treated mice (FIG. 4C). Some treated mice were still alive at >200 days (6.5 months), so the upper limit of survival after a single treatment is not yet known. AAV-hSPL treated KO mice remained active and behaved identically to WT and heterozygous controls until a few days prior to the onset of weight loss. Hypoalbuminemia (reduced albumin in blood/serum), albuminuria (increased albumin in urine) and the gross pallor observed in KO kidneys all showed delayed onset in AAV-hSPL treated mice (FIG. 4D-G). Whereas lymphopenia persisted in treated SPL KO pups, anemia responded to AAV-hSPL treatment (FIG. 4H). These cumulative findings demonstrate that administration of AAV-hSPL to newborn SPL KO pups increases SPL expression and activity in some tissues, partially normalizes S1P and other sphingolipids, improves weight gain, extends mean survival and corrects some SPLIS and SPL KO mouse phenotypes. The delayed progression of kidney disease observed in AAV-hSPL treated KO mice suggests that: 1) normalizing the circulating S1P suffices to attenuate kidney pathology; 2) improving bioavailability to kidney may improve efficacy of AAV-hSPL; 3) the KO lethality may be due to adrenal failure, not nephrosis; 4) AAV-hSPL expression may be compromised with time.

A single dose of 10-20 μL AAV-hSPL solution (3.5×10¹⁰ vg/μL) was given IV into facial vein of SPL KO pups. Mice were sacrificed at DOL 21 and tissues harvested.

FIG. 3A. Left shows WB of kidney (Kid) and adrenal gland (AG) probed with anti-mSPL (which detects endogenous mSPL in HET and cross-reacts with hSPL in AAV-treated mice). A faint band is detected in kidney. GAPDH is a loading control. Right shows WB of liver probed with hSPL-specific antibody.

FIG. 3B. SPL activity in WT, KO and AAV-hSPL treated KO liver.

FIG. 3C. qRT-PCR of hSPL in AAV-hSPL treated KO mouse tissues.

FIG. 3D,E. Plasma (FIG. 3D) and liver sphingolipids (FIG. 3E) in WT, KO and AAV-hSPL-treated KO (AAV) mice. P<0.05.

FIG. 3F. C14 and 16 ceramides in liver samples from WT, KO and treated mice.

FIG. 4A. Weight gain in KO (inverted triangle), WT (upright triangle) and AAV-hSPL treated KO (diamond) pups.

FIG. 4B. Image of 22-day old WT and KO on left, WT and AAV-hSPL treated KO on right.

FIG. 4C. Survival of WT, KO and AAV-hSPL treated KO mice.

FIG. 4D. Serum albumin levels in WT, KO and early vs. late AAV-hSPL treated mice showing delayed disease progression.

FIG. 4E. Coomassie gel of urinary proteins. Albumin standards S2=0.2 μg; S5=15 μg. Prominent albumin band in 1 untreated KO at 3 wks age and reduced albumin in the treated KO at 8, 9 and 11 wks.

FIG. 4F. Urine albumin levels corresponding to gel. Note increase over time in 1 treated KO over 3 time points.

FIG. 4G. Images of WT, KO and AAV-treated KO kidneys over time showing pallor in the KO kidneys and delayed disease progression in treated kidneys.

FIG. 4H. Blood cell indices show absolute lymphopenia (Abs Lymph), low % lymphocytes (% Lymph) and anemia with low red blood cell (RBC) counts, hemoglobin (HB) and hematocrit (HCT) in KO pups compared to WT. Anemia corrects but lymphopenia persists in AAV-hSPL treated KO mice. For each the plotted indices, bars from left to right correspond to WT (1^{st} bar), KO (2^{nd} bar), and AAV KO (3^{rd} bar) pups.

### Example 4: Treatment of Newborn Sgpl1 KO Mice with AAV-SPL Dramatically Prolongs Survival

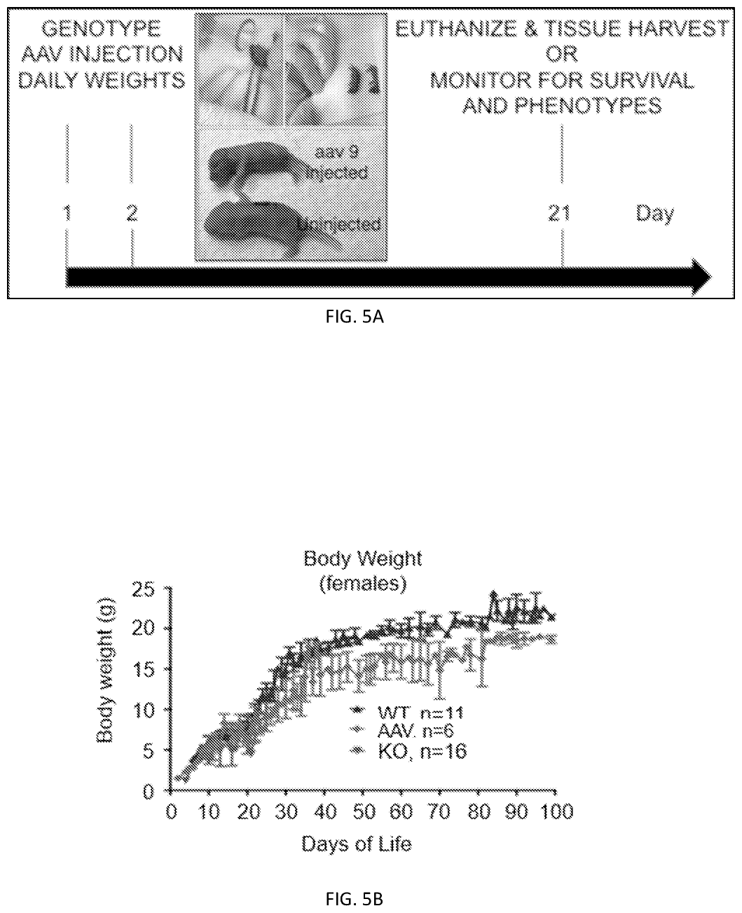

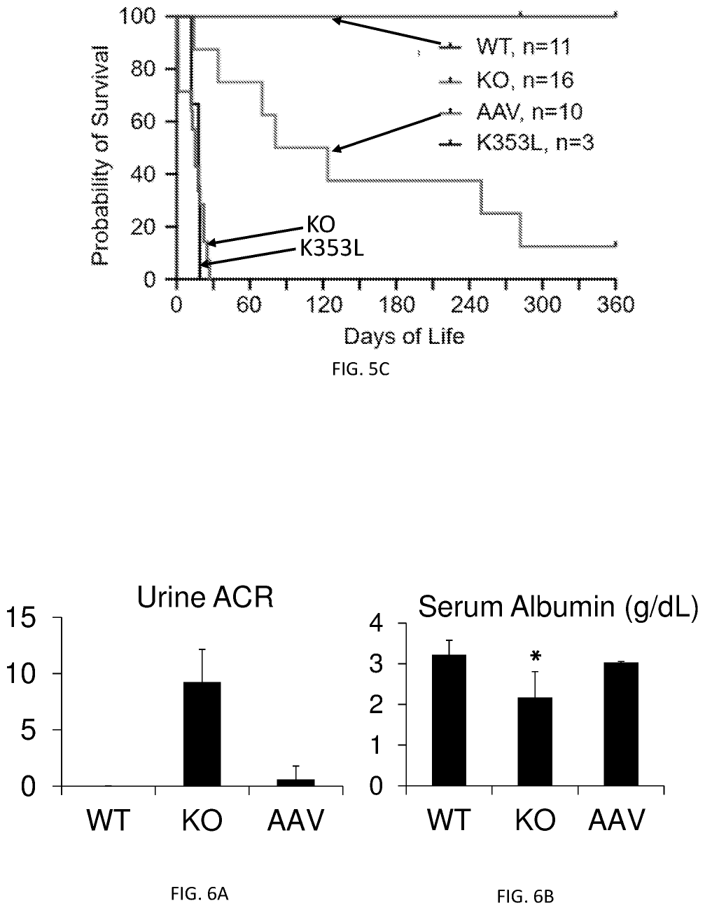

To further test the impact of SGPL1 gene replacement on Sgpl1 KO mouse survival, AAV-SPL was delivered to ten newborn Sgpl1 KO pups using the strategy shown in FIG. 5A. Litters produced from heterozygous matings were genotyped on day of life (DOL) 1 by toe biopsy. Sgpl1 KO pups were treated at 1-2 DOL with a single dose of approximately 7×10¹¹ vg by intravenous (IV) injection. An additional three KO pups were injected with the same dose of AAV-SPL^{K353L} Pups were monitored for weight gain and euthanized when the humane endpoint was reached. As shown in FIG. 4B, AAV-SPL treated Sgpl1 KO mice were appreciably larger than untreated Sgpl1 KO mice. Sgpl1 KO mice treated with AAV-SPL gained weight steadily throughout their lives, as shown in FIG. 5B. Mean survival increased from 14.6 days in untreated KO controls to 125 days in treated mice, with some mice living for 8-11 months (FIG. 5C). In contrast, mice treated with catalytically inactive AAV-SPL^{K353L} showed no improvement in weight gain or survival, with a mean survival of 15 days (FIG. 5C). Theses finding demonstrate that introduction of a catalytically active human SPL into SgPl1 KO pups significantly prolongs their survival.

FIGS. 5A-5C. Treatment of newborn Sgp1 KO mice with AAV-SPL prolongs survival. FIG. 5A. Treatment and euthanasia schedule. FIG. 5B. Weight gain of female WT (top most curve), untreated Sgpl1 KO (bottom most curve) and AAV-SPL treated KO (middle) mice during the first three months of life. FIG. 5D. Kaplan-Meier survival curve for WT, untreated Sgpl1 KO, AAV-SPL treated KO and AAV-SPL^{K353L} treated KO mice.

### Example 5: AAV-SPL Treatment of Sgpl1 KO Mice Prevents the Development of SPLIS Nephrosis

Renal involvement is present in 80% of SPLIS cases and is a major cause of morbidity, hospitalizations, surgical intervention (renal transplantation) and death. Similarly, Sgpl1 KO mice develop nephrosis with high urine albumin to creatinine ratio (ACR) and low serum albumin levels, accompanied by pathological changes consistent with podocyte and glomerular injury prior to their demise at the time of weaning (at 21 DOL). The first few Sgpl1 KO mice treated with AAV-SPL lived for 35-70 days and exhibited higher serum albumin levels than untreated Sgp1 KO mice (data not shown). This indicated that AAV-SPL treatment may have delayed the progression of nephrosis but did not completely prevent it. In contrast, mice treated later lived for 8-11.5 months and showed normal ACR (FIG. 6A) and normal serum albumin levels (FIG. 6B) up to the time of their deaths. Periodic acid-Schiff stained sections of kidney cortices from untreated Sgp1 KO mice showed glomeruli that were enlarged, with a wide size distribution and mesangial expansion compared to those of WT kidneys (data not shown). These features were absent in the kidney cortices of Sgp1 KO mice treated with AAV-SPL, but were present in those of Sgp1 KO mice that were treated with catalytically inactive AAV-SPL^{K353L} (data not shown). Some glomeruli of untreated Sgp1 KO mice and Sgpl1 KO mice treated with AAV-SPL^{K353L} exhibited evidence of sclerosis, whereas no glomeruli from WT and AAV-SPL treated Sgpl1 KO mice exhibited focal sclerosis.

FIGS. 6A-6B. AAV-SPL treatment prevents the development of SPLIS nephrosis. FIG. 6A. Urine albumin/creatinine ratio in wild type (WT, n=3), untreated Sgpl1 knockout (KO, n=3) and AAV-SPL treated KO (AAV, n=3) mice. FIG. 6B. Serum albumin levels in WT, KO and AAV-SPL treated KO mice. *p<0.05 for KO vs. WT. There was no significant difference between AAV vs. WT.

### Example 6: Stat3 Activation, Inflammatory and Fibrogenic Cytokine Upregulation in SPLIS Kidneys

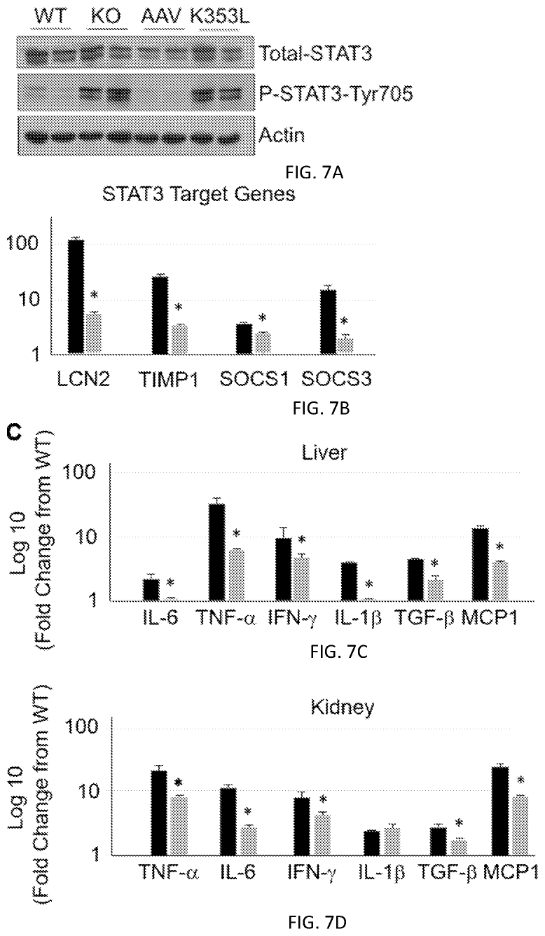

Signal transducer and activator of transcription (STAT3) is a transcription factor that contributes to carcinogenesis and the regulation of inflammation, autophagy, cellular metabolism and mitochondrial function. It can be activated by various cytokines through binding and activation of gp130, the IL-6 receptor. Both glomerular injury and S1P signaling have been associated with activation of the STAT3 inflammatory signaling pathway in mice and humans (H. Lee et al., Nat Med 16, 1421-1428 (2010). Immunoblotting was used to compare the phosphorylated (active) and total STAT3 protein levels in Sgpl1 WT and KO mouse kidney homogenates. As shown in FIG. 7A, Sgpl1 KO kidneys exhibited activation of STAT3 in comparison to WT controls. STAT3 activation was not detected in AAV-SPL treated KO mouse kidneys, whereas kidneys of KO mice treated with AAV-SPL^{K323L} were not protected from STAT3 activation. Gene expression of STAT3 target genes suppressor of cytokine signaling 1 and 3 (SOCS1, SOCS3) and two STAT3 target genes implicated in various forms of kidney injury, namely lipocalin 2 (LCN2) and tissue inhibitor of metalloprotease 1 (TIMP1), were measured by qRT-PCR in kidney tissues from each group. STAT3 target genes were upregulated in untreated Sgpl1 KO kidney tissue compared to WT, while kidneys of AAV-SPL treated KO mice showed lower STAT3 target gene elevation compared to untreated Sgpl1 KO mice (FIG. 7B). Pro-inflammatory cytokine elevation in the liver tissues of Sgp1 KO mice was previously reported (38). We confirmed that the pro-inflammatory cytokine and STAT3 pathway activator IL-6 was upregulated in Sgp1 KO liver tissues (FIG. 7C). Additional pro-inflammatory cytokines including TNF-α, IFN-γ, IL-1β and MCP1 were also elevated in Sgpl1 KO liver compared to WT liver (FIG. 7C). The cytokine transforming growth factor beta (TGFβ) is a well-characterized inducer of fibrosis. IGFβ levels were also elevated in Sgp1 KO liver compared to WT liver (FIG. 7C). Importantly, each of these pro-inflammatory and fibrogenic cytokines was also elevated in the kidneys of the Sgpl1 KO mice compared to WT (FIG. 7D). Further, with the exception of IL-1β, AAV-SPL treatment diminished the levels of pro-inflammatory and fibrogenic cytokines in Sgp1 KO kidneys (FIG. 7D). These cumulative findings demonstrate that AAV-SPL treatment in the newborn period prevents the development of nephrosis, STAT3 activation and pro-inflammatory and fibrogenic cytokine upregulation in Sgpl1 KO mice.

FIGS. 7A-7D. STAT3 activation and cytokine upregulation in SPLIS kidneys. FIG. 7A. Immunoblot showing abundance of total and Tyrosine 705-phosphorylated (activated) STAT3 in the kidney tissues of wild type (WT), untreated Sgpl1 knockout (KO), AAV-SPL treated KO (AAV) and AAV-SPL^{K353L} treated KO (K353L) mice. Actin is a loading control. FIG. 7B. Relative expression of STAT3 target genes lipocalin 2 (LCN2), tissue inhibitor of metalloprotease 1 (TIMP1), and suppressor of cytokine signaling 1 and 2 (SOCS1, SOCS2) in Sgpl1 KO mice (black bars) and AAV-SPL treated KO mice (gray bars), shown as log fold change from WT. FIG. 7C, liver and FIG. 7D, kidney pro-inflammatory and fibrogenic cytokines in Sgp1 KO mice (black bars) and AAV-SPL treated KO mice (gray bars), shown as log fold change from WT. *p<0.05 for AAV vs. KO.

### Example 7: AAV-SPL Treatment Prevents Neurodevelopmental Delay in Pre-Weaned Sgpl1 KO Mice

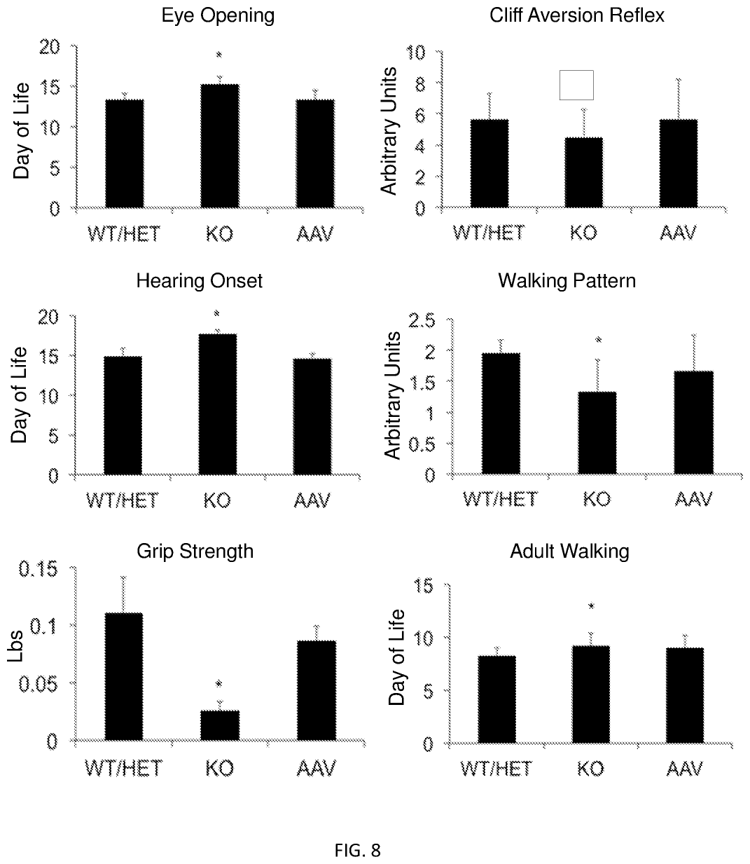

In nearly half of SPLIS cases, neurological manifestations are present, including sensorineural hearing loss, cranial nerve defects, a Charcot-Marie-Tooth type peripheral neuropathy, seizures, ataxia and developmental delay or regression. Brain-specific conditional Sgpl1 KO mice live normal lifespans but exhibit motor and behavioral abnormalities as adults (D. N. Mitroi et al., Sci Rep 6, 37064 (2016). However, characterization of the neurodevelopmental function of global Sgpl1 KO pups prior to their demise at weaning has not been reported. A battery of neurological tests designed to quantitatively measure the achievement of basic neurodevelopmental milestones in pre-weaned mice was used to compare AAV-SPL treated and untreated Sgpl1 KO pups and WT littermate controls. Sgpl1 KO pups showed significant delays in the achievement of eye opening, hearing onset, and adult pattern walking (FIG. 8). In addition, grip strength was weaker in Sgp1 KO compared to WT pups, indicating a motor function deficit. Cliff aversion reflex scores were lower in KO pups compared to WT littermates, but the difference was not statistically significant. Other tests, including paw grasping reflex, righting reflex, negative geotaxis and inverted clinging obstacle tests, were either highly variable from day to day or did not reveal defects in the Sgpl1 KO pups (data not shown). In contrast, the neurodevelopmental scores in AAV-SPL treated KO pups were not significantly different from WT, indicating no evidence of developmental delay (FIG. 8). These results indicate that neurological impairment is manifest in Sgp1 KO mice as early as the first days and weeks of life, and that immediate treatment after birth with AAV-SPL gene replacement prevents neurodevelopmental delay.

FIG. 8. Sgpl1 KO mice exhibit developmental delay, which is prevented by AAV-SPL. A battery of six neurodevelopmental milestones were scored in untreated wild type (WT; n=8), heterozygous (HET; n=14), Sgpl1 knockout (KO; n=6) and AAV-SPL treated Sgpl1 KO (AAV; n=3) pups. *p≤0.003 for KO vs. WT and HET groups combined (WT/HET). There was no significant difference between AAV and WT/HET or between WT and HET for any of the tests.

### Example 8: Sgpl1 KO Mice Exhibit Glucocorticoid Deficiency that is not Averted by AAV-SPL

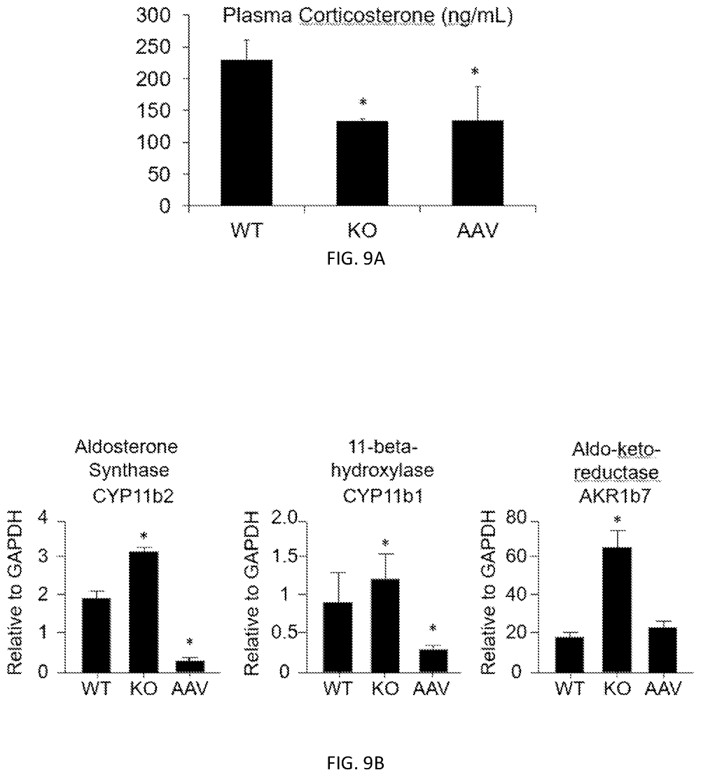

Approximately seventy percent of SPLIS patients exhibit glucocorticoid hormone deficiency as a manifestation of primary adrenal insufficiency. However, blood steroid hormone measurements in Sgpl1 KO mice have not been reported. Plasma levels of corticosterone, the main glucocorticoid hormone found in mice, were measured in treated and untreated Sgpl1 KO mice and WT littermate controls. Samples were collected from all subjects at the corticosterone peak of the circadian cycle. As shown in FIG. 9A, Sgpl1 KO mice exhibited significantly lower corticosterone levels than WT levels regardless of treatment group. Thus, Sgpl1 KO mice exhibit adrenal insufficiency which appears refractory to treatment. To gain additional insight into the impact of AAV-SPL on adrenal gland function, the gene expression of CYP11b1, CYP11b2 and AKR1b7 was measured—which respectively encode the three adrenal cortical enzymes 11-beta-hydroxyase, aldosterone synthase and aldo-keto-reductase—in the adrenal tissues of treated and untreated Sgpl1 KO and WT littermate mice (FIG. 9B). CYBP11b1 and CYP11b2 are involved in adrenal hormone synthesis, whereas AKR1b7 is involved in lipid peroxide detoxification (A. D. Borowsky et al., J Lipid Res 53, 1920-1931 (2012). Interestingly, all three genes were upregulated in untreated Sgpl1 KO adrenal glands, whereas levels were either suppressed (CYP11b1, CYP11b2) or normalized compared to WT mice in the adrenal glands of AAV-SPL treated KO mice. These findings indicate that Sgpl1 KO mice exhibit adrenal insufficiency with reactive upregulation of cortical gene expression. SGPL1 gene replacement reduces cortical gene expression to WT levels or below but does not restore normal corticosterone levels under the conditions tested.

FIGS. 9A-9B. Sgpl1 KO mice exhibit glucocorticoid deficiency. FIG. 9A. Corticosterone levels were measured by radioimmunoassay in the plasma of wild type (WT), untreated Sgpl1 knockout (KO) and AAV-SPL treated KO (AAV) mice. All subjects were euthanized at weaning at the time of peak glucocorticoid levels (lights out). *p<0.01 for KO vs. WT; p<0.02 for AAV vs. WT. There was no significant difference between AAV and KO. FIG. 9B. Expression levels of three genes of the adrenal cortex, CYP11b1, CYP11b2 and AKR1b7, were measured in adrenal gland tissues of WT, KO and AAV mice by qRT-PCR. *p<0.03 for KO vs. WT and AAV vs. WT.

### Example 9: AAV-SPL Treatment Corrects High Cholesterol Levels in Sgpl1 KO Mile

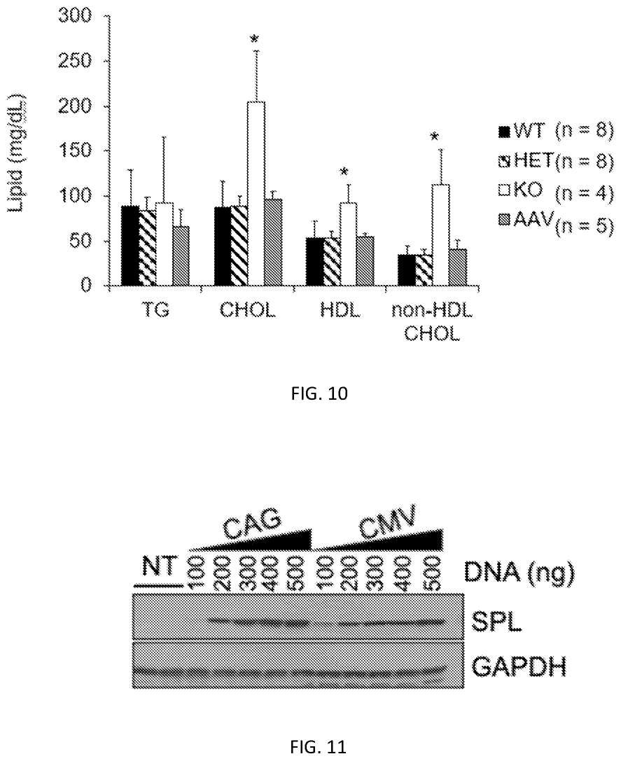

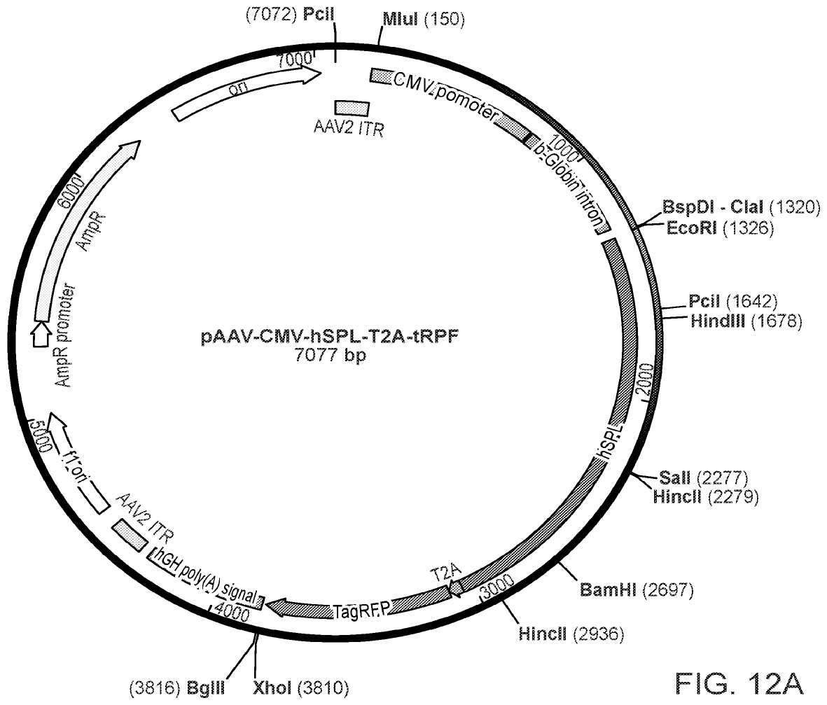

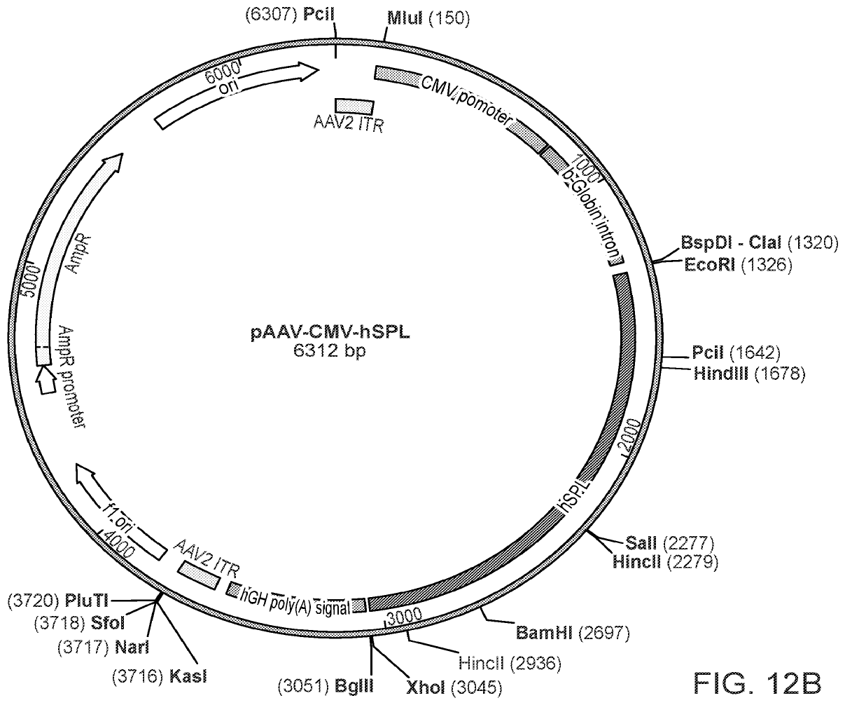

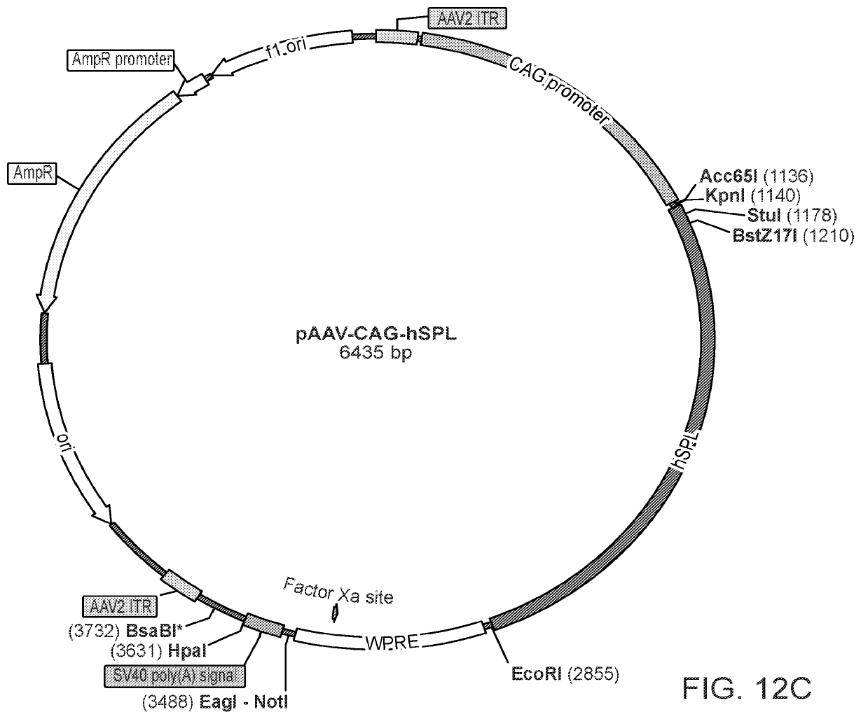

Sgpl1 KO mice were previously shown to exhibit high circulating cholesterol levels including total, free, and esterified cholesterol (M. Bektas et al., Journal of Biological Chemistry 285, 10880-10889 (2010). Low-density lipoprotein (LDL) and high-density lipoprotein (HDL) cholesterol were also substantially increased in KO compared to WT blood. Comparing plasma of WT, HET, KO and AAV-SPL treated KO mice at the time of weaning, significantly higher levels of total cholesterol, as well as higher HDL and non-HDL cholesterol in KO plasma compared to WT or HET plasma was confirmed, whereas triglyceride levels were not appreciably different in any of the groups (FIG. 10). AAV-SPL treated mice plasma had total cholesterol, HDL cholesterol and non-HDL cholesterol levels that were no different than WT and HET controls. Therefore, the impact of SPL disruption on circulating cholesterol was completely prevented by SGPL1 gene replacement in the newborn period.

FIG. 10. Plasma lipids of treated and untreated Sgpl1 KO mice. Plasma triglycerides (TG), total cholesterol (CHOL), high density lipid cholesterol (HDL) and non-HDL cholesterol (non-HDL CHOL), the latter representing low, intermediate and very low-density lipid, were measured in the plasma of untreated wild type (WT), untreated Sgpl1 knockout (KO) and AAV-SPL treated Sgpl1 knockout (AAV) mice. All mice were euthanized at 28 days of life. *p<0.007 for KO vs. WT and for KO vs. AAV.

### Example 10: SPL Expression from CAG Promoter

Effect of the promoter on expression of SPL was tested by comparing CMV and CAG promoters. FIG. 11 that shows that the CAG promoter is at least as strong if not stronger than the CMV promoter when transfected into HEK293 cells in a head-to-head comparison of SPL expression (based on DNA amount transfected).

The tomato gene in pAAV-CAG-dtTomato (Addgene #59462) was replaced with hSPL. Then, 100-500 ng of the resulting plasmid or original (CMV-driven) AAV-SPL were transfected into HEK293 cells to compare SPL expression. GAPDH is used as a loading control. NT=no treatment.

pAAV-CAG-hSPL sequence is set forth in
SEQ ID NO: 6.
(SEQ ID NO: 6)
CTGCGCGCTCGCTCGCTCACTGAGGCCGCCCGGGCA
AAGCCCGGGCGTCGGGCGACCTTTGGTCGCCCGGCC
TCAGTGAGCGAGCGAGCGCGCAGAGAGGGAGTGGCC
AACTCCATCACTAGGGGTTCCTTGTAGTTAATGATT
AACCCGCCATGCTACTTATCTACGTAGCCATGCTCT
AGGAAGATCGTACCATTGACGTCAATAATGACGTAT
GTTCCCATAGTAACGCCAATAGGGACTTTCCATTGA
CGTCAATGGGTGGAGTATTTACGGTAAACTGCCCAC
TTGGCAGTACATCAAGTGTATCATATGCCAAGTACG
CCCCCTATTGACGTCAATGACGGTAAATGGCCCGCC
TGGCATTATGCCCAGTACATGACCTTATGGGACTTT
CCTACTTGGCAGTACATCTACGTATTAGTCATCGCT
ATTACCATGGTCGAGGTGAGCCCCACGTTCTGCTTC
ACTCTCCCCATCTCCCCCCCCTCCCCACCCCCAATT
TTGTATTTATTTATTTTTTAATTATTTTGTGCAGCG
ATGGGGGCGGGGGGGGGGGGGGGGCGCGCGCCAGGC
GGGGCGGGGCGGGGCGAGGGGCGGGGCGGGGCGAGG
CGGAGAGGTGCGGCGGCAGCCAATCAGAGCGGCGCG
CTCCGAAAGTTTCCTTTTATGGCGAGGCGGCGGCGG
CGGCGGCCCTATAAAAAGCGAAGCGCGCGGCGGGCG
GGAGTCGCTGCGACGCTGCCTTCGCCCCGTGCCCCG
CTCCGCCGCCGCCTCGCGCCGCCCGCCCCGGCTCTG
ACTGACCGCGTTACTCCCACAGGTGAGCGGGCGGGA
CGGCCCTTCTCCTCCGGGCTGTAATTAGCGCTTGGT
TTAATGACGGCTTGTTTCTTTTCTGTGGCTGCGTGA
AAGCCTTGAGGGGCTCCGGGAGGGCCCTTTGTGCGG
GGGGAGCGGCTCGGGGCTGTCCGCGGGGGGACGGCT
GCCTTCGGGGGGGACGGGGCAGGGCGGGGTTCGGCT
TCTGGCGTGTGACCGGCGGCTCTAGAGCCTCTGCTA
ACCATGTTCATGCCTTCTTCTTTTTCCTACAGCTCC
TGGGCAACGTGCTGGTTATTGTGCTGTCTCATCATT
TTGGCAAAGAATTGGATCCGGTACCGCCACCATGGC
GAGCACAGACCTTCTGATGTTGAAGGCCTTTGAGCC
CTACTTAGAGATTTTGGAAGTATACTCCACAAAAGC
CAAGAATTATGTAAATGGACATTGCACCAAGTATGA
GCCCTGGCAGCTAATTGCATGGAGTGTCGTGTGGAC
CCTGCTGATAGTCTGGGGATATGAGTTTGTCTTCCA
GCCAGAGAGTTTATGGTCAAGGTTTAAAAAGAAATG
TTTTAAGCTCACCAGGAAGATGCCCATTATTGGTCG
TAAGATTCAAGACAAGTTGAACAAGACCAAGGATGA
TATTAGCAAGAACATGTCATTCCTGAAAGTGGACAA
AGAGTATGTGAAAGCTTTACCCTCCCAGGGTCTGAG
CTCATCTGCTGTTTTGGAGAAACTTAAGGAGTACAG
CTCTATGGACGCCTTCTGGCAAGAGGGGAGAGCCTC
TGGAACAGTGTACAGTGGGGAGGAGAAGCTCACTGA
GCTCCTTGTGAAGGCTTATGGAGATTTTGCATGGAG
TAACCCCCTGCATCCAGATATCTTCCCAGGACTACG
CAAGATAGAGGCAGAAATCGTGAGGATAGCTTGTTC
CCTGTTCAATGGGGGACCAGATTCGTGTGGATGTGT
GACTTCTGGGGGAACAGAAAGCATACTGATGGCCTG
CAAAGCATATCGGGATCTGGCCTTTGAGAAGGGGAT
CAAAACTCCAGAAATTGTGGCTCCCCAAAGTGCCCA
TGCTGCATTTAACAAAGCAGCCAGTTACTTTGGGAT
GAAGATTGTGCGGGTCCCATTGACGAAGATGATGGA
GGTGGATGTGCGGGCAATGAGAAGAGCTATCTCCAG
GAACACTGCCATGCTCGTCTGTTCTACCCCACAGTT
TCCTCATGGTGTAATAGATCCTGTCCCTGAAGTGGC
CAAGCTGGCTGTCAAATACAAAATACCCCTTCATGT
CGACGCTTGTCTGGGAGGCTTCCTCATCGTCTTTAT
GGAGAAAGCAGGATACCCACTGGAGCACCCATTTGA
TTTCCGGGTGAAAGGTGTAACCAGCATTTCAGCTGA
CACCCATAAGTATGGCTATGCCCCAAAAGGCTCATC
ATTGGTGTTGTATAGTGACAAGAAGTACAGGAACTA
TCAGTTCTTCGTCGATACAGATTGGCAGGGTGGCAT
CTATGCTTCCCCAACCATCGCAGGCTCACGGCCTGG
TGGCATTAGCGCAGCCTGTTGGGCTGCCTTGATGCA
CTTCGGTGAGAACGGCTATGTTGAAGCTACCAAACA
GATCATCAAAACTGCTCGCTTCCTCAAGTCAGAACT
GGAAAATATCAAAGGCATCTTTGTTTTTGGGAATCC
CCAATTGTCAGTCATTGCTCTGGGATCCCGTGATTT
TGACATCTACCGACTATCAAACCTGATGACTGCTAA
GGGGTGGAACTTGAACCAGTTGCAGTTCCCACCCAG
TATTCATTTCTGCATCACATTACTACACGCCCGGAA
ACGAGTAGCTATACAATTCCTAAAGGACATTCGAGA
ATCTGTCACTCAAATCATGAAGAATCCTAAAGCGAA
GACCACAGGAATGGGTGCCATCTATGGCATGGCCCA
GACAACTGTTGACAGGAATATGGTTGCAGAATTGTC
CTCAGTCTTCTTGGACAGCTTGTACAGCACCGACAC
TGTCACCCAGGGCAGCCAGATGAATGGTTCTCCAAA
ACCCCACTGAGAATTCGATATCAAGCTTATCGATAA
TCAACCTCTGGATTACAAAATTTGTGAAAGATTGAC
TGGTATTCTTAACTATGTTGCTCCTTTTACGCTATG
TGGATACGCTGCTTTAATGCCTTTGTATCATGCTAT
TGCTTCCCGTATGGCTTTCATTTTCTCCTCCTTGTA
TAAATCCTGGTTGCTGTCTCTTTATGAGGAGTTGTG
GCCCGTTGTCAGGCAACGTGGCGTGGTGTGCACTGT
GTTTGCTGACGCAACCCCCACTGGTTGGGGCATTGC
CACCACCTGTCAGCTCCTTTCCGGGACTTTCGCTTT
CCCCCTCCCTATTGCCACGGCGGAACTCATCGCCGC
CTGCCTTGCCCGCTGCTGGACAGGGGCTCGGCTGTT
GGGCACTGACAATTCCGTGGTGTTGTCGGGGAAATC
ATCGTCCTTTCCTTGGCTGCTCGCCTGTGTTGCCAC
CTGGATTCTGCGCGGGACGTCCTTCTGCTACGTCCC
TTCGGCCCTCAATCCAGCGGACCTTCCTTCCCGCGG
CCTGCTGCCGGCTCTGCGGCCTCTTCCGCGTCTTCG
CCTTCGCCCTCAGACGAGTCGGATCTCCCTTTGGGC
CGCCTCCCCGCATCGATACCGTCGACCCGGGCGGCC
GCTTCGAGCAGACATGATAAGATACATTGATGAGTT
TGGACAAACCACAACTAGAATGCAGTGAAAAAAATG
CTTTATTTGTGAAATTTGTGATGCTATTGCTTTATT
TGTAACCATTATAAGCTGCAATAAACAAGTTAACAA
CAACAATTGCATTCATTTTATGTTTCAGGTTCAGGG
GGAGATGTGGGAGGTTTTTTAAAGCAAGTAAAACCT
CTACAAATGTGGTAAAATCGATAAGGATCTTCCTAG
AGCATGGCTACGTAGATAAGTAGCATGGCGGGTTAA
TCATTAACTACAAGGAACCCCTAGTGATGGAGTTGG
CCACTCCCTCTCTGCGCGCTCGCTCGCTCACTGAGG
CCGGGCGACCAAAGGTCGCCCGACGCCCGGGCTTTG
CCCGGGCGGCCTCAGTGAGCGAGCGAGCGCGCAGCT
GCATTAATGAATCGGCCAACGCGCGGGGAGAGGCGG
TTTGCGTATTGGGCGCTCTTCCGCTTCCTCGCTCAC
TGACTCGCTGCGCTCGGTCGTTCGGCTGCGGCGAGC
GGTATCAGCTCACTCAAAGGCGGTAATACGGTTATC
CACAGAATCAGGGGATAACGCAGGAAAGAACATGTG
AGCAAAAGGCCAGCAAAAGGCCAGGAACCGTAAAAA
GGCCGCGTTGCTGGCGTTTTTCCATAGGCTCCGCCC
CCCTGACGAGCATCACAAAAATCGACGCTCAAGTCA
GAGGTGGCGAAACCCGACAGGACTATAAAGATACCA
GGCGTTTCCCCCTGGAAGCTCCCTCGTGCGCTCTCC
TGTTCCGACCCTGCCGCTTACCGGATACCTGTCCGC
CTTTCTCCCTTCGGGAAGCGTGGCGCTTTCTCATAG
CTCACGCTGTAGGTATCTCAGTTCGGTGTAGGTCGT
TCGCTCCAAGCTGGGCTGTGTGCACGAACCCCCCGT
TCAGCCCGACCGCTGCGCCTTATCCGGTAACTATCG
TCTTGAGTCCAACCCGGTAAGACACGACTTATCGCC
ACTGGCAGCAGCCACTGGTAACAGGATTAGCAGAGC
GAGGTATGTAGGCGGTGCTACAGAGTTCTTGAAGTG
GTGGCCTAACTACGGCTACACTAGAAGAACAGTATT
TGGTATCTGCGCTCTGCTGAAGCCAGTTACCTTCGG
AAAAAGAGTTGGTAGCTCTTGATCCGGCAAACAAAC
CACCGCTGGTAGCGGTGGTTTTTTTGTTTGCAAGCA
GCAGATTACGCGCAGAAAAAAAGGATCTCAAGAAGA
TCCTTTGATCTTTTCTACGGGGTCTGACGCTCAGTG
GAACGAAAACTCACGTTAAGGGATTTTGGTCATGAG
ATTATCAAAAAGGATCTTCACCTAGATCCTTTTAAA
TTAAAAATGAAGTTTTAAATCAATCTAAAGTATATA
TGAGTAAACTTGGTCTGACAGTTACCAATGCTTAAT
CAGTGAGGCACCTATCTCAGCGATCTGTCTATTTCG
TTCATCCATAGTTGCCTGACTCCCCGTCGTGTAGAT
AACTACGATACGGGAGGGCTTACCATCTGGCCCCAG
TGCTGCAATGATACCGCGAGACCCACGCTCACCGGC
TCCAGATTTATCAGCAATAAACCAGCCAGCCGGAAG
GGCCGAGCGCAGAAGTGGTCCTGCAACTTTATCCGC
CTCCATCCAGTCTATTAATTGTTGCCGGGAAGCTAG
AGTAAGTAGTTCGCCAGTTAATAGTTTGCGCAACGT
TGTTGCCATTGCTACAGGCATCGTGGTGTCACGCTC
GTCGTTTGGTATGGCTTCATTCAGCTCCGGTTCCCA
ACGATCAAGGCGAGTTACATGATCCCCCATGTTGTG
CAAAAAAGCGGTTAGCTCCTTCGGTCCTCCGATCGT
TGTCAGAAGTAAGTTGGCCGCAGTGTTATCACTCAT
GGTTATGGCAGCACTGCATAATTCTCTTACTGTCAT
GCCATCCGTAAGATGCTTTTCTGTGACTGGTGAGTA
CTCAACCAAGTCATTCTGAGAATAGTGTATGCGGCG
ACCGAGTTGCTCTTGCCCGGCGTCAATACGGGATAA
TACCGCGCCACATAGCAGAACTTTAAAAGTGCTCAT
CATTGGAAAACGTTCTTCGGGGCGAAAACTCTCAAG
GATCTTACCGCTGTTGAGATCCAGTTCGATGTAACC
CACTCGTGCACCCAACTGATCTTCAGCATCTTTTAC
TTTCACCAGCGTTTCTGGGTGAGCAAAAACAGGAAG
GCAAAATGCCGCAAAAAAGGGAATAAGGGCGACACG
GAAATGTTGAATACTCATACTCTTCCTTTTTCAATA
TTATTGAAGCATTTATCAGGGTTATTGTCTCATGAG
CGGATACATATTTGAATGTATTTAGAAAAATAAACA
AATAGGGGTTCCGCGCACATTTCCCCGAAAAGTGCC
ACCTAAATTGTAAGCGTTAATATTTTGTTAAAATTC
GCGTTAAATTTTTGTTAAATCAGCTCATTTTTTAAC
CAATAGGCCGAAATCGGCAAAATCCCTTATAAATCA
AAAGAATAGACCGAGATAGGGTTGAGTGTTGTTCCA
GTTTGGAACAAGAGTCCACTATTAAAGAACGTGGAC
TCCAACGTCAAAGGGCGAAAAACCGTCTATCAGGGC
GATGGCCCACTACGTGAACCATCACCCTAATCAAGT
TTTTTGGGGTCGAGGTGCCGTAAAGCACTAAATCGG
AACCCTAAAGGGAGCCCCCGATTTAGAGCTTGACGG
GGAAAGCCGGCGAACGTGGCGAGAAAGGAAGGGAAG
AAAGCGAAAGGAGCGGGCGCTAGGGCGCTGGCAAGT
GTAGCGGTCACGCTGCGCGTAACCACCACACCCGCC
GCGCTTAATGCGCCGCTACAGGGCGCGTCCCATTCG
CCATTCAGGCTGCGCAACTGTTGGGAAGGGCGATCG
GTGCGGGCCTCTTCGCTATTACGCCAG.

### Materials and Methods

Vector cloning, production and packaging: Human WT SGPL1 cDNA, hSPLK353L cDNA, and hSPLtRFP (D. R. Herr et al. Development 130, 2443-2453 (2003)) were separately cloned into EcoRI/XhoI sites in pAAV-MCS (Agilent Technologies, La Jolla, Calif.), which contains the CMV promoter/enhancer and AAV2 inverted terminal repeats. Inserts were confirmed by DNA sequence analysis. AAV virus was packaged with different serotype capsids to generate AAV8-, AAV9-, and AAV-PHP.eB-SPL virus. AAV virus was packaged using an adenovirus-free system in which AAV-SPL, pHelper, and AAV-RC plasmids pUCmini-iCAP-PHP.eB (Addgene plasmid #103005) were co-transfected into HEK293 cells (ATCC, Manassas, Va.). Virus was harvested and purified via iodixanol gradient ultracentrifugation (S. Zolotukhin et al., Gene Ther 6, 973-985 (1999)). After validating activity in cell culture experiments described below, AAV9-hSPL and AAV9-hSPLK353L virions were prepared at large scale for in vivo use. Virus titers were quantified by Taqman qPCR of ITR primers, and both probes were determined to be ˜3.5×10¹⁰ vector genomes (vg) per μL.

Functional testing of virus in vitro: Virus particles expressing wild type hSPL, hSPL and RFP, or catalytically inactive mutant SPLK353L proteins were used to infect immortalized human skin fibroblasts derived from a SPLIS patient generated as we described previously (S. Saygili et al., Pediatr Nephrol 34, 77-79 (2019)). Human samples were obtained with informed consent in accordance with a Benioff Children's Hospital Oakland approved institutional review board (IRB) protocol.

Immunoblotting: Proteins were extracted from fibroblasts or mouse tissue as described (P. Zhao et al., J Inherit Metab Dis, (2020)). The following antibodies were used: goat anti-human SGPL1 polyclonal antibody (AF5535, R&D Systems), anti-mouse Sgpl1 polyclonal antibody (Reiss et al, JBC), rabbit anti-GAPDH (sc-25778, Santa Cruz Biotechnology), anti-phosphorylated and total STAT3 (Santa Cruz Biotechnology) HRP conjugated second antibody was used to detected the signal by using SuperSignal West-Pico kit (Fisher Scientific, Rockford, Ill.). Radiographic bands were quantified and normalized using NIH ImageJ.

SPL activity assays: SPL activity was quantified in whole cell extracts and homogenized tissue by measuring the formation of the (2E)-hexadecenal product by quantification of a hydrazine derivative of the product by liquid chromatography-tandem mass spectrometry (LC-MS/MS) separation with multiple reaction monitoring essentially as described (J. H. Suh, et al., A facile stable-isotope dilution method for determination of sphingosine phosphate lyase activity. Chem Phys Lipids 194, 101-109 (2016)).

Animals: SPL global KO mice exhibit no SPL expression or activity and accumulate bioactive sphingolipids (M. Bektas et al., Journal of Biological Chemistry 285, 10880-10889 (2010)). The KO allele was generated using the ROSAFARY gene trap vector (J. Schmahl, et al., Nat Genet 39, 52-60 (2007), J. Schmahl, et al., Genes Dev 22, 3255-3267 (2008)). Het mice are viable and fertile and are crossed to obtain homozygous progeny. The KO is born at the expected Mendelian frequency and is indistinguishable at birth from its WT and het littermates. The line was backcrossed to C57BL/6 background for 10 generations and colonies are refreshed by backcrossing every 3-4 generations. Sgpl1 global KO mice were bred and maintained as heterozygotes in an AAALAC Accredited animal facility with an automated light/dark cycle of 7 am/7 pm. Pups were genotyped on the first day of life by toe biopsy and using primers: SPL-F: CGC TCA GAA GGC TCT GAG TCA TGG (SEQ ID NO:7), SPL wt-R: CCA AGT GTA CCT GCT AAG TTC CAG (SEQ ID NO:8); SPL ko-R: CAT CAA GGA AAC CCT GGA CTA CTG (SEQ ID NO:9). All mouse studies were conducted in accordance with an approved University of California San Francisco Institutional Animal Care and Use Committee protocol.

AAV treatments: Homozygous knockout pups were anesthetized with isoflurane and injected with virus at a dose of 3.5-7.0×10¹¹ vector genomes in a volume of 10-20 microliters in sterile saline into the superficial temporal vein using a 33-gauge needle and a Yale Model YA-12 syringe pump. To aid temporal vein injection, the vector solution contained dye (1% food coloring). Successfully injected mice turn completely blue. Previous experience showed that the dye does not interfere with vector function or harm the pup and is excreted within 24 h. In some cases, virus solution was instead injected into the liver parenchyma as described (R. J. Chandler, et al., Hum Gene Ther 19, 53-60 (2008)). Injected mice were monitored and weighted every 3 days, and subjected to 24 h urine collections in a metabolic chamber, serial neurobehavioral examinations and/or phlebotomy at various time points. Survival study mice were euthanized when they reached the humane endpoint. Mice used for lipid measurements and bioavailability studies were euthanized at 21-28 days of life.

Laboratory analyses and ACR calculations: Serum albumin and urine albumin and creatinine were measured using the COBAS Integra 400 Plus instrument (Roche Diagnostics; Indianapolis, Ind.). To measure urine creatinine, creatininase, creatinase, and sarcosine oxidase are used in succession to liberate H₂O₂, which then reacts with 4-aminophenazone and 2,4,6-triido-3-hydroxybenzoic acid to form a quinone imine chromogen which is measured colorimetrically. Both serum and urine albumin measurements involve the reaction of anti-albumin antibodies with the sample antigen. Resulting antigen/antibody complexes are then measured turbidimetrically according to the manufacturer's instructions (Roche Diagnostics).

Cholesterol measurements: Plasma triglycerides, total cholesterol, and HDL cholesterol, were measured by enzymatic endpoint analysis using enzyme reagent kits (AMS Diagnostics) on a clinical chemistry analyzer (Liasys 330, AMS Diagnostics).

Kidney pathology: Periodic acid Schiff staining of 3 μM sections of FFPE-fixed and paraffin embedded mouse kidneys was performed.

Corticosterone measurements: Corticosterone was measured in serum samples by RIA, using a commercial kit (MP Biomedicals; Orangeburg, N.Y.) at the University of Virginia Ligand Assay and Analysis Core (Charlottesville, Va.). The method was validated for mouse serum samples using a protocol based on the recommendations of the Endocrine Society's Sex Steroid Assays Reporting Task Force. The RIA evaluation included the following indices: accuracy, linearity, functional sensitivity, precision and correlation to a previous or established method. Assay characteristics were as follows: sensitivity=15 ng/ml; intra-assay coefficient of variation (CV)=6.3%; inter-assay CV=7.1%.

Statistical Analysis: For comparison of 2 groups of identical sample size, unpaired students t-test was performed. For groups of Ñ3, one-way ANOVA was used. P<0.05 was considered significant.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for treating SPL insufficiency syndrome (SPLIS) in a subject, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby treating the SPLIS in the subject.

2. The method of claim 1, wherein the subject has an increased level of plasma sphingosine-1-phosphate (S1P) and the administering results in at least a 5% reduction in plasma sphingosine-1-phosphate (S1P) in the subject.

3. The method of claim 1, wherein the subject has an increased level of C14 and 16 ceramides in the liver and the administering results in at least a 5% reduction in C14 and 16 ceramides in liver of the subject.

4. The method of claim 1, wherein the subject has hypoalbuminemia and the administering results in at least a 5% reduction in hypoalbuminemia in the subject.

5. The method of claim 1, wherein the subject has albuminuria and the administering results in at least a 5% reduction in albuminuria in the subject.

6. The method of claim 1, wherein the expression of SPL in the subject after the administering is at least 10% of the normal SPL level.

7. The method of claim 1, wherein the subject has lymphopenia and is diagnosed as having SPLIS based on sequencing of the SGPL1 gene.

8. The method of claim 7, wherein the subject is a newborn.

9. A method for preventing a subject from developing SPL insufficiency syndrome (SPLIS), the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of the SPL in the subject thereby preventing the subject from developing SPLIS.

10. The method of claim 9, wherein the subject has an inactivating mutation in SGPL1 gene.

11. The method of claim 9, wherein the subject is a fetus in utero, a newborn, an infant, a toddler, or a child.

12. The method of claim 9, wherein the expression of SPL in the subject after the administering is at least 10% of the normal SPL level.

13. A method for reducing circulating sphingosine-1-phosphate (S1P) level in a subject having increased circulating S1P level, the method comprising administering to the subject a therapeutically effective dose of a recombinant adeno-associated viral (rAAV) virion comprising a nucleic acid encoding sphingosine-1-phosphate lyase (SPL), wherein administering the rAAV virion results in expression of SPL in the subject thereby reducing the circulating S1P level in the subject.

14. The method of claim 13, wherein the subject has or is at risk for developing SPL insufficiency syndrome (SPLIS).

15. The method of claim 13, wherein the subject has inflammation.

16. The method of claim 13, wherein the subject has cancer.

17. The method of claim 13, wherein the subject has inflammatory bowel disease.

18. The method of claim 13, wherein the subject has kidney disease.

19. The method of any one of claims 1-18, wherein the rAAV virion comprises AAV serotype 9 capsid proteins.

20. The method of any one of claims 1-18, wherein the rAAV virion comprises AAV-PHP.eb virions.

21. The method of any one of claims 1-18, wherein the rAAV virion comprises AAV-PHP.S virions.

22. The method of any one of claims 1-21, wherein the nucleic acid encoding SPL is flanked by AAV inverted terminal repeats (ITRs).

23. The method of any one of claims 1-22, wherein the subject is a human and the SPL is a human SPL.

24. The method of any one of claims 1-23, wherein the administering comprises intravenous administering.

25. The method of any one of claims 1-23, wherein the administering comprises intrathecal administering.

26. A recombinant Adeno-associated viral (rAAV) vector comprising an AAV inverted terminal repeat, a promoter/enhancer, a nucleic acid sequence encoding sphingosine-1-phosphate lyase (SPL), and an AAV inverted terminal repeat.

27. The rAAV vector of claim 26, wherein the SPL is a human SPL.

28. The rAAV vector of claim 26, wherein the rAAV vector is AAV-PHP.B, AAV-PHP.eB, AAV-PHP.S or AAV-Anc80.

29. The rAAV vector of claim 26, wherein the rAAV vector is AAV-9 vector.

30. The rAAV vector of claim 26, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin—Globin (CAG) Hybrid Promoter, or albumin promoter.

31. A recombinant adeno-associated viral (rAAV) virion comprising a nucleic acid encoding sphingosine-1-phosphate lyase (SPL).

32. The rAAV virion of claim 31, comprising a rAAV vector comprising an AAV inverted terminal repeat, a promoter/enhancer, the nucleic acid sequence encoding sphingosine-1-phosphate lyase (SPL), and an AAV inverted terminal repeat.

33. The rAAV virion of claim 31, wherein the SPL is a human SPL.

34. The rAAV virion of claim 31, wherein the rAAV virion comprises AAV serotype 9 capsid proteins.

35. The rAAV virion of claim 31, wherein the rAAV virion comprises AAV-PHP.B, AAV-PHP.eB, AAV-PHP.S or AAV-Anc80 capsids.

36. The rAAV vector of claim 31, wherein the promoter is a cytomegalovirus (CMV) promoter, chicken β-actin promoter, human SGPL-1 gene promoter, human β-actin/CMV hybrid promoter, chicken β-actin/CMV hybrid promoter, CMV actin—Globin (CAG) Hybrid Promoter, or albumin promoter.

37. A composition comprising the rAAV vector of any one of claims 26-30 or the rAAV virion of any one of claims 31-36 and a pharmaceutically acceptable excipient.

38. The method of claim 1, wherein the therapeutically effective dose of the rAAV virion comprises a dose of about 10¹¹, 5×10¹¹, 10¹², 5×10¹², 10¹³, or 5×10¹³ viral genomes (vg).

39. A pharmaceutical composition comprising: the rAAV virion of claim 31 and a pharmaceutically acceptable diluent, carrier or excipient, wherein the composition comprises about 10¹¹, 5×10¹¹, 10¹², 5×10¹², 10¹³, or 5×10¹³ viral genomes (vg) of the rAAV virion.


SEQUENCE LISTING (9 sequences)

SEQ ID NO: 1 - protein, 568 aa, Homo sapiens
MPSTDLLMLKAFEPYLEILEVYSTKAKNYVNGHCTKYEPWQLIAWSVVWTLLIVWGYEFVFQPESLWSRFKKKCFKLTRKMPIIGRKIQDKLNKTKDDISKNMSFLKVDKEYVKALPSQGLSSSAVLEKLKEYSSMDAFWQEGRASGTVYSGEEKLTELLVKAYGDFAWSNPLHPDIFPGLRKIEAEIVRIACSLFNGGPDSCGCVTSGGTESILMACKAYRDLAFEKGIKTPEIVAPQSAHAAFNKAASYFGMKIVRVPLTKMMEVDVRAMRRAISRNTAMLVCSTPQFPHGVIDPVPEVAKLAVKYKIPLHVDACLGGFLIVFMEKAGYPLEHPFDFRVKGVTSISADTHKYGYAPKGSSLVLYSDKKYRNYQFFVDTDWQGGIYASPTIAGSRPGGISAACWAALMHFGENGYVEATKQIIKTARFLKSELENIKGIFVFGNPQLSVIALGSRDFDIYRLSNLMTAKGWNLNQLQFPPSIHFCITLLHARKRVAIQFLKDIRESVTQIMKNPKAKTTGMGAIYGMAQTTVDRNMVAELSSVFLDSLYSTDTVTQGSQMNGSPKPH

SEQ ID NO: 2 - DNA, 1704 bp, Homo sapiens
atgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccacac

SEQ ID NO: 3 - DNA, 2101 bp, Homo sapiens
acacacacacacagggcttcatcggctcagagagttgcaaacccagacccatgttctaggctgggctctgccaggaactgctaggagtcgcatctctctctggacctcagtttcttcacgtgtatttgtgtattctctggggtggagctggcgaatagctggagacccctctagccctaccattttttgattctaattaaccaaaaaggatattcgaggtccccgctacaaattctgaacccttggcttcccctccaaactcccacacaaactccaccccatcctgcctgtgtgtctttgggaggatcatttccttctttggtcttggtgtccttgtttacaggttgagggatgataagagtcacctgatctgggcaagtcaggccataaataaggcttgtatgtaaaggtgcctagcatagttcttggcaaagcaaggatcagtcgatatgattgcattggtttgagcatctgggaagaatgagcagactgcatactgtgtgttagggtgtagggaggaatcagacgtgaccctgccctctagtcatctgggtgcacagattctagattaaaggatgtgggggtggtaactaacaaggagatggagaggaggtgtgcagttcctgggggatctgatgaagaattaaagcagaagggtaggagatggagaatgggaaggcatacctgaaaccctaaagcaactcttgatgagtaggcattggcaggaggccccagaacattttctgaccctcaaacactgagaatgcatgtctgctggggaatagtagtgagggggctaagggtatgggggtgttcatgcctgagtcaggggctctggatggtagaaatgtctgggagttctgacggaatgggggtgaggaggcctaaagataacctgttcatagtctttcagggccttaaccattatggctggggaagtgggaagtgccgatggggtatgcagaggagggcagctgtgatagtacatttcatcctgaaagcactacatgtggataatgacggtagtgatgatgccctaattttagtgtcatggcaacattttccaaggggatggaattgggtaccctcattagtgtcatttttgtttgagagatttcaagagtctatctaggtgtcattttaaaatctattgaaaaaaaatcttgaatgcaggtcactgaccagttggttccatgtaactgcactgccttctgctaccactaaagcaatgttatgacaacctggacaagtcatgggaaacctatgtgagacttagttttttctttttctttttggagttgggggtctcactatgttgctcaggctgatctagagctcctgggctcagacaaccctcccgcctcagcatccccaagtgccgggattaccgatgtaagccactacacccagctgagacttagttttttcatctggaaatgaaggcgacctttgtgatcccagtgggcccttgcacttgtgtataaggtgattcccagcactgctcatgaaagtagtgatggcagagtagcctctgctctccattttatttttggggtgggggggggcagtggtggaggggaggatgaggatgatatttccattaaataagtttccttgttgcttttcttcaggcaccactgacctcgatggaaaaatgaagtccctggcccaggaaagagacccaactctgtggctgttttggattagtttgtacaatgccctgcagacctactctctcaggaggctccaagtgaccaaatgtgacaagaaagttttttgtccaaggcagtttggaagagaagttggtgaccgtcgggataccacagccgccccaggacggcctggttgagacattcactggagggtctgggtgcagcccgctgcctggccggtaggcggcgcgcacaggccgtggggcccgggtctgggcgtgcgcgcggctggtagcagcggggccgcgcacgccagggtccgggagccgggccggtgcccccggagccatttccgggaggggcgaggccggcggctgccgggcctccaatctcggcggcggcggcggcaacaggggagcctgggtctcgcggcctgcgagtccgtcgcg

SEQ ID NO: 4 - DNA, 7077 bp, Artificial sequence
  synthetic sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccacacgagggcagaggaagtcttctaacatgcggtgacgtggaggagaatcccggccctatggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 5 - DNA, 6312 bp, Artificial sequence
  synthetic sequence
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtggagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttgcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacatcgattgaattcgccaccatgcctagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaattgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 6 - DNA, 6435 bp, Artificial sequence
  synthetic sequence
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattggatccggtaccgccaccatggcgagcacagaccttctgatgttgaaggcctttgagccctacttagagattttggaagtatactccacaaaagccaagaattatgtaaatggacattgcaccaagtatgagccctggcagctaattgcatggagtgtcgtgtggaccctgctgatagtctggggatatgagtttgtcttccagccagagagtttatggtcaaggtttaaaaagaaatgttttaagctcaccaggaagatgcccattattggtcgtaagattcaagacaagttgaacaagaccaaggatgatattagcaagaacatgtcattcctgaaagtggacaaagagtatgtgaaagctttaccctcccagggtctgagctcatctgctgttttggagaaacttaaggagtacagctctatggacgccttctggcaagaggggagagcctctggaacagtgtacagtggggaggagaagctcactgagctccttgtgaaggcttatggagattttgcatggagtaaccccctgcatccagatatcttcccaggactacgcaagatagaggcagaaatcgtgaggatagcttgttccctgttcaatgggggaccagattcgtgtggatgtgtgacttctgggggaacagaaagcatactgatggcctgcaaagcatatcgggatctggcctttgagaaggggatcaaaactccagaaattgtggctccccaaagtgcccatgctgcatttaacaaagcagccagttactttgggatgaagattgtgcgggtcccattgacgaagatgatggaggtggatgtgcgggcaatgagaagagctatctccaggaacactgccatgctcgtctgttctaccccacagtttcctcatggtgtaatagatcctgtccctgaagtggccaagctggctgtcaaatacaaaataccccttcatgtcgacgcttgtctgggaggcttcctcatcgtctttatggagaaagcaggatacccactggagcacccatttgatttccgggtgaaaggtgtaaccagcatttcagctgacacccataagtatggctatgccccaaaaggctcatcattggtgttgtatagtgacaagaagtacaggaactatcagttcttcgtcgatacagattggcagggtggcatctatgcttccccaaccatcgcaggctcacggcctggtggcattagcgcagcctgttgggctgccttgatgcacttcggtgagaacggctatgttgaagctaccaaacagatcatcaaaactgctcgcttcctcaagtcagaactggaaaatatcaaaggcatctttgtttttgggaatccccaattgtcagtcattgctctgggatcccgtgattttgacatctaccgactatcaaacctgatgactgctaaggggtggaacttgaaccagttgcagttcccacccagtattcatttctgcatcacattactacacgcccggaaacgagtagctatacaattcctaaaggacattcgagaatctgtcactcaaatcatgaagaatcctaaagcgaagaccacaggaatgggtgccatctatggcatggcccagacaactgttgacaggaatatggttgcagaattgtcctcagtcttcttggacagcttgtacagcaccgacactgtcacccagggcagccagatgaatggttctccaaaaccccactgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccag

SEQ ID NO: 7 - DNA, 24 bp, Artificial sequence
  synthetic sequence
cgctcagaaggctctgagtcatgg

SEQ ID NO: 8 - DNA, 24 bp, Artificial sequence
  synthetic sequence
ccaagtgtacctgctaagttccag

SEQ ID NO: 9 - DNA, 24 bp, Artificial sequence
  synthetic sequence
catcaaggaaaccctggactactg